(19) United States
(12) Patent Application Publication
(10) Pub. No.: US 2022/0017594 A1
(43) Pub. Date: Jan. 20, 2022

(54) IL-2 DEPENDENT NK-92 CELLS WITH STABLE FC RECEPTOR EXPRESSION

(71) Applicant: NantKwest, Inc., San Diego, CA (US)

(72) Inventors: Francisco Navarro, San Diego, CA (US); Hans G. Klingemann, San Diego, CA (US); Laurent H. Boissel, San Diego, CA (US); Abhijit Dandapat, San Diego, CA (US)

(21) Appl. No.: 17/295,010

(22) PCT Filed: Nov. 25, 2019
(86) PCT No.: PCT/US2019/063069

§ 371 Date: May 18, 2021

Related U.S. Application Data
(60) Provisional application No. 62/771,479, filed on Nov. 26, 2018.

(51) Int. Cl. C07K 14/735; A61K 35/17; C12N 15/86; C12N 5/0783

(52) U.S. Cl. CPC C07K 14/70535; A61K 35/17; C12N 2740/15043; C12N 5/0646; C12N 15/86

(57) ABSTRACT

Provided herein are populations of IL2 Dependent haNK® cells, which express a high affinity CD16 but does not express IL-2. These cells maintain stable expression of Fc receptor CD16 while retaining cytotoxicity. In some embodiments, the expression level of CD16 decreases no more than 20% when the cells are activated as compared to expression level of CD16 on the cells before activation. Compositions and kits comprising the cells, and methods of making and using the IL2 Dependent haNK® cells are also provided.

### RELATED APPLICATION

This application claims the benefit of U.S. Provisional Application No. 62/771,479, filed on Nov. 26, 2018. The content of said provisional application is herein incorporated by reference in its entirety for all purposes.

### BACKGROUND

Anticancer treatment with monoclonal antibodies has significantly improved the clinical outcome in patients with cancer. One of the major mechanisms of action of therapeutic antibodies is through antibody-dependent cell-mediated cytotoxicity (ADCC). Natural killer cells could be used as cytotoxic effector cells for cell-based immunotherapy since they are a major effector cell for ADCC.

Referred to herein as “NK-92®” is a cytolytic cancer cell line which was discovered in the blood of a subject suffering from a non-Hodgkin's lymphoma and then immortalized ex vivo. NK-92® cells are derived from NK cells, but lack the major inhibitory receptors that are displayed by normal NK cells, while retaining the majority of the activating receptors. NK-92® cells do not, however, attack normal cells nor do they elicit an unacceptable immune rejection response in humans. Characterization of the NK-92® cell line is disclosed in WO 1998/49268 and U.S. Patent Application Publication No. 2002-0068044. NK-92® cells have also been evaluated as a potential therapeutic agent in the treatment of certain cancers.

Although NK-92® cells retain almost all of the activating receptors and cytolytic pathways associated with NK cells, they do not express CD16 on their cell surfaces. CD16 is an Fc receptor which recognizes and binds to the Fc portion of an antibody to activate NK cells for the ADCC effector mechanism. Because they lack CD16 receptors, unmodified NK-92® cells are unable to lyse target cells via the ADCC mechanism.

Natural NK cells express CD16, but the CD16 is susceptible to ADAM17-mediated proteolytic cleavage when the NK cells are activated by various stimuli. For example, it is known that co-culturing of NK cells with K562 tumor cells stimulates the CD16 cleavage protease, which leads to shedding of CD16 surface expression in NK cells. This rapid down-regulation of CD16 in NK cells following activation significantly impairs the ADCC activity of the NK cells.

### BRIEF SUMMARY

Provided herein are populations of modified NK-92® cells, compositions and kits comprising the cells, and methods of making and using the populations of cells. The modified NK-92® cells express CD16 (e.g., a high affinity variant of the Fc receptor CD16) and do not express IL-2. These modified NK-92® cells exhibit high level expression of CD16, and the expression level is maintained during and/or after activation by stimulants, target cell engagement, or ADCC. This stable expression of CD16 allows the modified NK-92® cells to effect serial killing of the target cells during and/or ADCC. The exclusion of the IL-2 transgene from the modified NK-92® cells minimizes negative impact of IL-2 transgene and allows the flexibility of introducing additional transgenes that can confer desired properties to the modified NK-92®. For example, IL-2 might be released in vivo due to cell leakage or cell death. IL-2 may promote recruitment and expansion of Tregs, causing immunosuppression. High doses of IL-2 have been shown to induce strong side effects in patients, for example, increased risk of infection, bruising and bleeding, fatigue, etc. See https://www.cancerresearchuk.org/about-cancer/cancer-in-general/treatment/cancer-drugs/drugs/aldesleukin/side-effects. Omitting IL-2 from the NK-92® cells would avoid these adverse effects.

The modified NK-92® cells described above are herein referred to as “IL2 Dependent CD16 Positive NK-92® cells” or “IL2 Dependent haNK® cells.”

In some embodiments, the disclosure provides a population of modified NK-92® cells expressing CD16 (SEQ ID NO:1), wherein the modified NK-92® cells do not express IL-2, and wherein the population comprises one or more of the modified NK-92® cells. The modified NK-92® cells may comprise a nucleic acid of CD16 (SEQ ID NO:2). In some embodiments, the modified NK-92® cells have ADCC.

In some embodiments, the expression level of CD16 of the modified NK-92® cells decreases no more than 20% when the cells are activated as compared to expression level of CD16 on the cells before activation. In some embodiments, the percentage of cells that are positive for CD16 decreases no more than 10% after the cells are contacted with the target cells as compared to the cells before the contact.

In some embodiments, the modified NK-92® cells exhibit no reduction or a reduction in CD16 expression of no more than 20% after activation, and wherein the modified NK-92® cells maintain a steady state of cytotoxicity for at least 5 hours from the initiation of the activation.

In some embodiments, the cells express higher level of CD16 than NK cells from a donor. In some embodiments, the expression of CD16 is measured by flow cytometry. In some embodiments, the percentage of cells that are positive for CD16 decreases no more than 20% after the cells are activated as compared to the cells before activation. In some embodiments, the cells are activated by one or more compounds selected from the group consisting of PMA, ionomycin, and LPS. In some embodiments, the modified NK-92® cells are activated by phytohemagglutinin (PHA), an innate pathway activation via co-incubation with K562 cells or byADCC via co-incubation with Rituxan and DOHH.

In some embodiments, the population of modified NK-92® cells are activated by contacting target tumor cells. The target tumor cells may be cells selected from the group consisting of K562 cells and SKBR-3 cells. In some embodiments, the CD16 expression of the population of modified NK-92® cells that have been activated decreases no more than 10% as compared to the modified NK-92® cells before the activation. In some embodiments, the expression level of CD16 on the NK-92® cells that have been activated decreases no more than 5% as compared to the expression level of CD16 on the modified NK-92® cells before the activation.

In some embodiments, the population of modified NK-92® cells are activated by contacting an antibody and a target cell, wherein the incubation results in ADCC. In some embodiments, the antibody is anti-CD20 antibody and the target cell is a DOHH-2 cell. In some embodiments, the antibody is an anti-HER2 antibody and the target cell is a SKBR3 cell. In some embodiments, the ratio of the number of modified NK-92® cells to the number of target cells is within a range from 1:1 to 1:10, end points inclusive. In some embodiments, the population of modified NK-92® cells of any of claims 1-18, wherein the modified NK-92® cells have direct cytotoxicity of at least 60% when the effector to target ratio of the cytotoxicity assay is 5:1. In some embodiments, the modified NK-92® cells have ADCC activity of at least 40%.

In some embodiments, the modified NK-92® cells additionally express a chimeric antigen receptor.

In some embodiments, the modified NK-92® cells additionally express a suicide gene. In some embodiments, the suicide gene is selected from the group consisting of a thymidine kinase (TK) gene, a Cytosine deaminase, cytochrome P450, and iCas9.

In some embodiments, the disclosure provides a method of producing a population of modified NK-92® cells that are capable of maintaining expression of CD16 during activation, wherein the method comprises introducing CD16 (SEQ ID NO:2), but not IL-2, into NK-92® cells, wherein the expression of CD16 on the activated modified NK-92® cells is no less than 80% of the CD16 expression on the modified NK-92® cells before the activation. In some embodiments, the introduction of CD16 is through lentiviral infection.

In some embodiments, the disclosure also provides a kit comprising the population of cells of any of the embodiments described above. In some embodiments, the kit further comprises an antibody.

In some embodiments, the disclosure also provides a pharmaceutical composition comprising the population of cells of any of the embodiments described above and a pharmaceutically acceptable excipient. In some embodiments, the disclosure provides a method of treating a subject comprising administering to the subject a pharmaceutical composition described herein.

### BRIEF DESCRIPTION OF THE DRAWINGS

FIGS. 1A and 1B show the results of flow cytometric analysis of CD16 expression on “haNK 003 cells” (IL-2 independent NK-92® cells that express CD16), IL2 Dependent haNK® cells, and donor NK cells, before (FIG. 1A) and after (FIG. 1B) PMA/ionomycin treatment.

FIGS. 2A-2C show results of flow cytometric analysis of CD16 expression on haNK003 cells, IL2 Dependent haNK® cells and donor NK cells. FIG. 2A shows CD16 expression on the cells before co-culturing with the K562 cells, FIG. 2B shows CD16 expression after co-culturing with K562 cells for 4 hours, and FIG. 2C shows CD16 expression after co-culturing for 24 hours.

FIGS. 3A and 3B show CD16 expression level in haNK003 cells and IL2 Dependent haNK® cells after ADCC. ADCC was performed by co-culturing haNK® and DoHH cells in presence of 1 μg/ml rituximab for 4 hours at E:T ratio of 1:0 (effectors alone) to 1:4, then CD16 expression level was measured at 4 hours and 24 hours by flow cytometry. FIG. 3A shows the flow cytometric analysis of CD16 expression level in haNK-003 and IL2 Dependent haNK® after ADCC along with control (E:T=1:0). FIG. 3B shows the median fluorescence intensity (MFI) of CD16 expression after 4 hour and 24 hours.

FIG. 4 shows the median fluorescence intensity (MFI) of CD16 surface staining of haNK003 cells and IL2 Dependent haNK® clones (H2, H7, H20, P74, P82, and P110) at various time points within a period of 24 weeks following the infection of aNK™ cells with lentivirus carrying a CD16 transgene.

FIG. 5A and FIG. 5B show the lysis of K562 cells by aNK™ cells, haNK003 cells and IL2 Dependent haNK® cells when the NK-92® cells are mixed with K562 cells at different effector-to-target ratios.

FIGS. 6A and 6B show the antibody-dependent cell-mediated cytotoxicity (ADCC) of IL2 Dependent haNK® cells (FIG. 6A: H clones and FIG. 6B: P clones) on the SKBR-3 cells in the presence of Herceptin at an effector-to-target ratio of 10:1. The Y axis values were determined by subtracting the percentage of SKBR-3 cells lysed by IL2 Dependent haNK® cells in the presence of isotype control antibody from the percentage of SKBR-3 cells lysed by IL2 Dependent haNK® cells in the presence of Herceptin under the same conditions.

### DETAILED DESCRIPTION

Provided herein are modified NK-92® cells, i.e., IL2 Dependent haNK® cells expressing a high affinity variant of the Fc receptor CD16 and are therefore capable of CD16 targeted antibody-dependent cell-mediated cytotoxicity (ADCC). The IL2 Dependent haNK® cells disclosed in this application do not express interleukin 2 (IL-2), e.g., human IL-2 (GenBaNK™ Accession No.: AAH70338.1) or any polypeptide comprising the amino acid sequence of IL-2.

ADCC is mediated by recognition of the Fc fragment of the target-bound antibody (IgG) via the CD16 Fc receptor, which activates the modified NK-92® cells for targeted killing. ADCC is important for a number of therapeutic applications. For example, ADCC by the IL2 Dependent haNK® cells can be elicited by CD16 receptor binding to the Fc fragment of target cell-bound IgG to activate the IL2 Dependent haNK® cells for targeted killing.

In response to certain stimuli, CD16 is cleaved close to the cell membrane resulting in release of the extracellular portion of the receptor and down regulation of expression following activation (See, Jing, et al., PLOS one, 10(3):e0121788 DOI:10.1371/journal.pone.0121788 (2015)). Under normal conditions, this mechanism helps to control NK cell cytotoxicity, but in the tumor environment, this can reduce ADCC potency and cancer cell killing. Advantageously, the IL2 Dependent haNK® cells provided in this disclosure showed excellent ADCC activity against cancer cells, possibly and without limitation in theory due to the fact that the expression level of CD16 is maintained during and/or after ADCC. ADCC activity, with regard to the modified NK-92® cells disclosed herein, refers to the ability to kill target cells through ADCC. In one exemplary embodiment, ADCC activity can be determined by the formula: [% Killing in a reaction of E+T in the presence of mAB—% Killing in a reaction of E+T in the absence of mAb]/[100−% Killing in a reaction of E+T in the absence of mAb], where E refers to the modified NK-92® cells, T refers to the target cells, mAb refers to an antibody of interest, and % killing refers to the percentage of cells lysed in the reaction.

The IL2 Dependent haNK® cells provided in this disclosure are generated through stable transfection of NK-92® cells with a plasmid containing sequences for CD16, the high affinity Fc-gamma receptor (FcγRIIIa/CD16a), SEQ ID NO:1. The IL2 Dependent haNK® cells do not express IL-2. Accordingly, this disclosure provides a population of modified NK-92® cells, i.e., IL2 Dependent haNK® cells, having antibody-dependent cell-mediated cytotoxicity (ADCC) comprising nucleic acid molecules comprising CD16 (SEQ ID NO: 2).

Optionally, the modified NK-92® cells comprise a nucleic acid sequence with 70%, 80%, 90%, or 95% identity to SEQ ID NO: 2. Optionally, the modified NK-92® cells comprise a nucleic acid sequence with 90%, 91%, 92%, 93%, 94%, 95%, 96%, 97%, 98% or 99% identity to SEQ ID NO:2. Optionally, the modified NK-92® cells comprise a polypeptide with 70%, 80%, 90%, or 95% identity to SEQ ID NO:1. Optionally, the modified NK-92® cells comprise a polypeptide with 90%, 91%, 92%, 93%, 94%, 95%, 96%, 97%, 98% or 99% identity to SEQ ID NO:1.

### Terminology

Nucleic acid, as used herein, refers to deoxyribonucleotides or ribonucleotides and polymers and complements thereof. The term includes deoxyribonucleotides or ribonucleotides in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, conservatively modified variants of nucleic acid sequences (e.g., degenerate codon substitutions) and complementary sequences can be used in place of a particular nucleic acid sequence recited herein. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A nucleic acid is operably linked when it is placed into a functional relationship with another nucleic acid sequence. For example, DNA that encodes a presequence or secretory leader is operably linked to DNA that encodes a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are near each other, and, in the case of a secretory leader, contiguous and in reading phase. However, enhancers do not have to be contiguous. For example, a nucleic acid sequence that is operably linked to a second nucleic acid sequence is covalently linked, either directly or indirectly, to such second sequence, although any effective three-dimensional association is acceptable. A single nucleic acid sequence can be operably linked to multiple other sequences. For example, a single promoter can direct transcription of multiple RNA species. Linking can be accomplished by ligation at convenient restriction sites. If such sites do not exist, the synthetic oligonucleotide adaptors or linkers are used in accordance with conventional practice.

The term “polypeptide,” as used herein, generally has its art-recognized meaning of a polymer of at least three amino acids and is intended to include peptides and proteins. However, the term is also used to refer to specific functional classes of polypeptides, such as, for example, desaturases, elongases, etc. For each such class, the present disclosure provides several examples of known sequences of such polypeptides. Those of ordinary skill in the art will appreciate, however, that the term polypeptide is intended to be sufficiently general as to encompass not only polypeptides having the complete sequence recited herein (or in a reference or database specifically mentioned herein), but also to encompass polypeptides that represent functional fragments (i.e., fragments retaining at least one activity) of such complete polypeptides. Moreover, those in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term polypeptide as used herein. Those in the art can determine other regions of similarity and/or identity by analysis of the sequences of various polypeptides described herein. As is known by those in the art, a variety of strategies are known and tools are available for performing comparisons of amino acid or nucleotide sequences to assess degrees of identity and/or similarity. These strategies include, for example, manual alignment, computer assisted sequence alignment and combinations thereof. A number of algorithms (which are generally computer implemented) for performing sequence alignment are widely available, or can be produced by one of skill in the art. Representative algorithms include, e.g., the local homology algorithm of Smith and Waterman (Adv. Appl. Math., 1981, 2: 482); the homology alignment algorithm of Needleman and Wunsch (J. Mol. Biol., 1970, 48: 443); the search for similarity method of Pearson and Lipman (Proc. Natl. Acad. Sci. (USA), 1988, 85: 2444); and/or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.). Readily available computer programs incorporating such algorithms include, for example, BLASTN, BLASTP, Gapped BLAST, PILEUP, CLUSTALW, etc. When utilizing BLAST and Gapped BLAST programs, default parameters of the respective programs may be used. Alternatively, the practitioner may use non-default parameters depending on his or her experimental and/or other requirements (see for example, the Web site having URL www.ncbi.nlm.nih.gov).

The term “transformation,” as used herein refers to a process by which an exogenous or heterologous nucleic acid molecule (e.g., a vector or recombinant nucleic acid molecule) is introduced into a recipient cell or microorganism. The exogenous or heterologous nucleic acid molecule may or may not be integrated into (i.e., covalently linked to) chromosomal DNA making up the genome of the host cell or microorganism. For example, the exogenous or heterologous polynucleotide may be maintained on an episomal element, such as a plasmid. Alternatively or additionally, the exogenous or heterologous polynucleotide may become integrated into a chromosome so that it is inherited by daughter cells through chromosomal replication. Methods for transformation include, but are not limited to, calcium phosphate precipitation; fusion of recipient cells with bacterial protoplasts containing the recombinant nucleic acid; treatment of the recipient cells with liposomes containing the recombinant nucleic acid; DEAE dextran; fusion using polyethylene glycol (PEG); electroporation; magnetoporation; biolistic delivery; retroviral infection; lipofection; and micro-injection of DNA directly into cells.

The term “transformed,” as used in reference to cells, refers to cells that have undergone transformation as described herein such that the cells carry exogenous or heterologous genetic material (e.g., a recombinant nucleic acid). The term transformed can also or alternatively be used to refer to microorganisms, strains of microorganisms, tissues, organisms, etc. that contain exogenous or heterologous genetic material.

The terms “modified” and “recombinant” when used with reference to a cell, nucleic acid, polypeptide, vector, or the like indicates that the cell, nucleic acid, polypeptide, vector or the like has been modified by or is the result of laboratory methods and is non-naturally occurring. Thus, for example, modified cells include cells produced by or modified by laboratory methods, e.g., transformation methods for introducing nucleic acids into the cell. Modified cells can include nucleic acid sequences not found within the native (non-recombinant) form of the cells or can include nucleic acid sequences that have been altered, e.g., linked to a non-native promoter.

As used herein, the term “effector-to-target ratio” refers to the ratio of the number of effector cells (e.g., NK-92® cells, such as IL2 Dependent haNK® cells) to the number of the target cells (e.g., tumor cells) used in an assay to assess the cytotoxicity of the effector cells on the target cells.

As used herein, “natural killer (NK) cells” are cells of the immune system that kill target cells in the absence of a specific antigenic stimulus, and without restriction according to major histocompatibility complex (MHC) class. Target cells may be cancer or tumor cells. NK cells are characterized by the presence of CD56 and the absence of CD3 surface markers.

As used herein, “NK-92® cells” refer to natural killer cells derived from the highly potent unique cell line described in Gong et al. (1994), rights to which are owned by NantKwest.

For purposes of this invention and unless indicated otherwise, the term “NK-92®” or “NK92” is intended to refer to the original NK-92® cell lines as well as NK-92® cell lines, clones of NK-92® cells, and NK-92® cells that have been modified (e.g., by introduction of exogenous genes). NK-92® cells and exemplary and non-limiting modifications thereof are described in U.S. Pat. Nos. 7,618,817; 8,034,332; 8,313,943; 9,181,322; 9,150,636; and published U.S. application Ser. No. 10/008,955, all of which are incorporated herein by reference in their entireties, and include wild type NK-92®, NK-92®-CD16, NK-92®-CD16-γ, NK-92®-CD16-ζ, NK-92®-CD16(F176V), NK-92® MI, and NK-92® CI. NK-92® cells are known to persons of ordinary skill in the art, to whom such cells are readily available from NantKwest, Inc. As used herein, the term “aNK™ cells” refers to unmodified natural killer cells derived from the highly potent unique cell line described in Gong et al. (1994), rights to which are owned by NantKwest. As used herein, the term “haNK® cells” refers to natural killer cells derived from the highly potent unique cell line described in Gong et al. (1994), rights to which are owned by NantKwest, modified to express CD16 on the cell surface (hereafter, “CD16 Positive NK-92® cells” or “haNK® cells”). Thus, examples of haNK® cells include IL2 Dependent haNK® cells (“haNK003 cells”) and IL2 Dependent haNK® cells the former additionally express recombinant IL-2 and the latter do not.

As used herein, the term “NK cells” refer to a) donor derived NK cells, b) NK-92.176V-CD16.ERIL2 cells (i.e., IL2 Independent haNK® cells) and c) NK-92.176V-CD16 cells (i.e., IL2 Dependent haNK® cells). As disclosed herein, donor derived NK cells exhibit a rapid and profound reduction of CD16 expression upon activation, with only a marginal recovery in expression after overnight recovery, haNK® cells (IL2 dependent and independent alike) exhibit little to no reduction in CD16 expression while maintaining peak cytotoxic potency.

The term “Fc receptor” refers to a protein found on the surface of certain cells (e.g., natural killer cells) that contribute to the protective functions of the immune cells by binding to part of an antibody known as the Fc region. Binding of the Fc region of an antibody to the Fc receptor (FcR) of a cell stimulates phagocytic or cytotoxic activity of a cell via antibody-mediated phagocytosis or antibody-dependent cell-mediated cytotoxicity (ADCC). FcRs are classified based on the type of antibody they recognize. For example, Fc-gamma receptors (FcγR) bind to the IgG class of antibodies. FcγRIII-A (also called CD16) is a low affinity Fc receptor bind to IgG antibodies and activate ADCC. FcγRIII-A are typically found on NK cells. NK-92® cells do not express FcγRIII-A. A representative amino acid sequence encoding CD16 is shown in SEQ ID NO: 1. A representative polynucleotide sequence encoding CD16 is shown in SEQ ID NO: 2. The complete sequences of CD16 can be found in the SwissProt database as entry P08637.

As used herein, the term “activation” with reference to the modified NK-92® cells or NK cells disclosed herein, refers to the phenomenon that NK cells are stimulated to perform cytotoxic function by contacting one or more activation agents (stimulants). These cytotoxic function may include releasing cytoplasm proteins, such as perforin and proteases known as granzymes, to induce apoptosis or lysis of the cells in close proximity. These activation agents include, but not limited to, various cytokines (e.g., interferons or macrophage-derived cytokines), plant lectins, (e.g., phytohemagglutinin (PHA), Concanavalin A (Con A), and pokeweed mitogen (PWM)), lipopolysaccharide (LPS), PMA (Phorbol 12-myristate 13-acetate)/ionomycin, purified protein derivative of tuberculin (PPD). Activation may refer to a) PHA stimulation, b) innate pathway activation via co-incubation with K562 or c) ADCC activation via co-incubation with Rituxan and DOHH.

In some embodiments, the activation agents may be tumor cells. In some embodiments, the activation agents are tumor cells that have ligands (e.g., ULBP and MICA/B), which can be recognized by receptors on NK cells or the modified NK-92® cells, e.g., NKG2D, NKp46, NKp30, and DNAM-1. This interaction activates the NK cells, which lyse the tumor cells. In some embodiments, the tumor cells that activate the NK cells or the modified NK-92® cells are K562 cells.

NK cells or the modified NK-92® cells can also be activated by contacting one or more activation agents comprising an antibody and its target cells. The Fc receptor CD16 expressed on NK cells or modified NK-92® cells recognizes and interacts with the Fc fragment of the target-bound antibody and this interaction activates the NK cells to lysis the target cells, a process known as the ADCC.

The term “expression” refers to the production of a gene product. The term “stable” when referred to expression means a polynucleotide is incorporated into the genome of the cell and expressed.

As used herein, the term “antibody” refers to an immunoglobulin or fragment thereof. The antibody may be of any type (e.g., IgG, IgA, IgM, IgE or IgD). Preferably, the antibody is IgG. An antibody may be non-human (e.g., from mouse, goat, or any other animal), fully human, humanized, or chimeric. An antibody may be polyclonal or monoclonal. Optionally, the antibody is monoclonal.

As used herein, the term “cancer” refers to all types of cancer, neoplasm, or malignant tumors found in mammals, including leukemia, carcinomas and sarcomas. Exemplary cancers include cancer of the brain, breast, cervix, colon, head & neck, liver, kidney, lung, non-small cell lung, melanoma, mesothelioma, ovary, sarcoma, stomach, uterus and medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

### NK-92® Cells

The NK-92® cell line is a unique cell line that was discovered to proliferate in the presence of interleukin 2 (IL-2). Gong et al., Leukemia 8:652-658 (1994). These cells have high cytolytic activity against a variety of cancers. The NK-92® cell line is a homogeneous cancerous NK cell population having broad anti-tumor cytotoxicity with predictable yield after expansion. Phase I clinical trials have confirmed its safety profile. NK-92® was discovered in the blood of a subject suffering from a non-Hodgkins lymphoma and then immortalized ex vivo. NK-92® cells are derived from NK cells, but lack the major inhibitory receptors that are displayed by normal NK cells, while retaining the majority of the activating receptors. NK-92® cells do not, however, attack normal cells nor do they elicit an unacceptable immune rejection response in humans. Characterization of the NK-92® cell line is disclosed in WO 1998/49268 and U.S. Patent Application Publication No. 2002-0068044.

The NK-92® cell line is found to exhibit the CD56bright, CD2, CD7, CD11a, CD28, CD45, and CD54 surface markers. It furthermore does not display the CD1, CD3, CD4, CD5, CD8, CD10, CD14, CD16, CD19, CD20, CD23, and CD34 markers. Growth of NK-92® cells in culture is dependent upon the presence of recombinant interleukin 2 (rIL-2), with a dose as low as 1 IU/mL being sufficient to maintain proliferation. IL-7 and IL-12 do not support long-term growth, nor do other cytokines tested, including IL-1α, IL-6, tumor necrosis factor α, interferon α, and interferon γ. NK-92® has high cytotoxicity even at a low effector:target (E:T) ratio of 1:1. Gong, et al., supra. NK-92® cells are deposited with the American Type Culture Collection (ATCC), designation CRL-2407.

Although NK-92® cells retain almost all of the activating receptors and cytolytic pathways associated with NK cells, they do not express CD16 on their cell surfaces. CD16 is an Fc receptor which recognizes and binds to the Fc portion of an antibody to activate NK cells for antibody-dependent cellular cytotoxicity (ADCC). Due to the absence of CD16 receptors, NK-92® cells are unable to lyse target cells via the ADCC mechanism and, as such, cannot potentiate the anti-tumor effects of endogenous or exogenous antibodies (i.e., Rituximab and Herceptin).

Studies on endogenous NK cells have indicated that IL-2 (1000 IU/mL) is critical for NK cell activation during shipment, but that the cells need not be maintained at 37° C. and 5% carbon dioxide. Koepsell, et al., Transfusion 53:398-403 (2013). However, endogenous NK cells are significantly different from NK-92® cells, in large part because of their distinct origins: NK-92® is a cancer-derived cell line, whereas endogenous NK cells are harvested from a donor (or the patient) and processed for infusion into a patient. Endogenous NK cell preparations are heterogeneous cell populations, whereas NK-92® cells are a homogeneous, clonal cell line. NK-92® cells readily proliferate in culture while maintaining cytotoxicity, whereas endogenous NK cells do not. In addition, an endogenous heterogeneous population of NK cells does not aggregate at high density. Furthermore, endogenous NK cells express Fc receptors, including CD-16 receptors that are not expressed by NK-92® cells.

### Producing IL2 Dependent Hank® Cells

IL2 Dependent haNK® cells disclosed in this application are NK-92® cells that are modified by introducing the high-affinity Fc gamma receptor (FcγRIIIa/CD16a) gene. This version of CD16 has a valine at amino acid 176, which has a high affinity for Fc fragment of antibodies and thus promotes increased ADCC.

The CD16 transgene can be engineered into an expression vector by any mechanism known to those of skill in the art. In some embodiments, the vector allows incorporation of the transgene(s) into the genome of the cell. In some embodiments, the vectors have a positive selection marker. Positive selection markers include any genes that allow the cell to grow under conditions that would kill a cell not expressing the gene. Non-limiting examples include antibiotic resistance, e.g., geneticin (Neo gene from Tn5).

Any number of vectors can be used to express the Fc receptors disclosed herein. In some embodiments, the vector is a plasmid. In one embodiment, the vector is a viral vector. Viral vectors include, but are not limited to, lentiviral vectors, retroviral vectors, adenoviral vectors, adeno-associated viral vectors, herpes simplex viral vectors, pox viral vectors, and others.

Transgenes can be introduced into the NK-92® cells using any transfection method known in the art, including, by way of non-limiting example, infection, electroporation, lipofection, nucleofection, or “gene-gun”.

In some embodiments, the CD16 transgene is introduced into NK-92® cells via a lentivirus. Typically the viral construct comprising the CD16 transgene is first introduced into a cell line with other plasmids that are required for packaging the lentiviruses. These plasmids may include at least a lentiviral packaging plasmid, e.g., pCMV-ΔR8.2 and an envelope plasmid, e.g., pCMV-VSV-G. After the transfection, the viral particles are formed in the culture supernatants. The supernatants are collected and used to infect NK-92® cells to produce the CD16-expressing, IL2 Dependent haNK® cells. In some embodiments, CD16-expressing cells are enriched before being plated by limited dilution. Individual clones of the CD-16 expressing cells can then be selected for expansion and then phenotypical and functional analyses.

Accordingly, provided in this disclosure is a population of modified NK-92® cells, i.e., IL2 Dependent haNK® cells, expressing CD16 (SEQ ID NO:1), wherein the modified NK-92® cells do not express IL-2, and wherein the population comprises one or more of the modified NK-92® cells. In some embodiments, the modified NK-92® cells comprises a nucleic acid of CD16 (SEQ ID NO:2). In some embodiments, the modified NK-92® cells have antibody-dependent cell-mediated cytotoxicity (ADCC).

The other type of haNK® cells, i.e., haNK003 cells, are produced through stable transfection by electroporation of NK-92® cells with a bicistronic plasmid-based vector containing sequences encoding CD16 (SEQ ID NO:1) and IL-2 (SEQ ID NO:3). The method of producing haNK003 is disclosed in application No. 62/468,890, the entire content of which is hereby incorporated by reference.

### Measuring CD16 Expression on IL2 Dependent Hank® Cells

Unlike NK cells, which loses expression of CD16 upon activation, IL2 Dependent haNK® cells provided in this disclosure are capable of maintaining high level of CD16 expression during and/or after activation. In general IL2 Dependent haNK® cells maintained high level of CD16 expression despite lacking IL-2 expression, indicating that IL-2 expression has no adverse effect on CD16 stability of haNK® cells.

CD16 expression level on haNK® cells, e.g., IL2 Dependent haNK® cells, can be measured by any of the methods known in the art to measure protein expression, for example, immunoblots, ELISAs, and flow cytometry. In some embodiments, CD16 expression is measured by flow cytometry. Typically detecting CD16 expression by flow cytometry involves incubating the cell sample with an anti-CD16 antibody that is conjugated to a fluorochrome. The sample is then analyzed on a flow cytometer to detect the bound antibody, and the intensity of the fluorochrome, e.g., the mean fluorescence intensity, from with bound antibody corresponds to the amount of the CD16 expression on the cells.

In some embodiments, the haNK® cells, e.g., the IL2 Dependent haNK® cells, are activated by incubating with PMA and ionomycin, and the CD16 expression level before and after the activation is measured. In some embodiments, the incubation lasts 0.5-4 hours, e.g., 0.5-2 hours, or about 1 hour. In some embodiments, the PMA used for activating haNK® cells is 10-80 nM, e.g., 20-60 nM, or about 40 nM. In some embodiments, the ionomycin used for activating the haNK™ cells is 200-1000 nM, e.g., 300-800 nM, 400-700 nM, or about 669 nM. In some embodiments, the expression level of CD16 on haNK™ cells decreases no more than 20%, e.g., no more than 40%, no more than 30%, no more than 25% as compared to the expression level of CD16 on the cells before activation. In some embodiments, the percentage of the haNK® cells that are positive for CD16 decreases, no more than 20%, or no more than 18%, after the cells are activated as compared to the cells before activation. In some embodiments, the percentage of the haNK® cells that are positive for CD16 does not decrease after activation. In some embodiments, haNK® cells (e.g., IL-2 dependent haNK® cells) that have been activated exhibit reduction in CD16 expression in the range of 0-20%, 0-10%, or 0-5%, as compared to CD16 expression level before the activation.

In some embodiments, the haNK® cells, e.g., IL2 Dependent haNK® cells, can be activated by co-culturing the haNK® cells with target cells (e.g., tumor cells) that are sensitive to NK cells. In some embodiments, the tumor cells are K562 cells. K562 cells are human chronic myelogenous leukemia cells. As used in this disclosure, the effector-to-target ratio refers to the number of effector cells (e.g., the NK-92® cells, including IL2 Dependent haNK® cells) to the number of the target cells. In some embodiments, the effector to target ratio is between 0.5:1 to 2:1, e.g., about 1:1. The incubation period typically has a length that is sufficient for complete cytotoxic killing of the target cells. In some embodiments, the incubation period is about 2 to 8 hours, e.g., about 4 hours. In some embodiments, following the incubation period, the cells are allowed to recover in culture medium. In some embodiments, the recovery period lasts 12-48 hours, e.g., about 20-28 hours, or about 24 hours. In some embodiments, the levels of CD16 expression on haNK® cells are monitored i) at the time before the cell are contacted with the target cells, e.g., target tumor cells, and ii) at the end of the incubation period and/or at the end of recovery period. In some embodiments, the CD16 expression of the population of haNK® cells after contacting with the target cells, e.g., at the end of the incubation period or at the end of the recovery period, decreases no more than 20%, no more than 10%, no more than 5%, no more than 3% as compared to the NK-92® cells before the activation. In some embodiments, the percentage of haNK® cells at the end of the incubation period or at the end of the recovery period that are positive for CD16 decreases no more than 20%, no more than 10% as compared to the cells before contacting the target cells.

In some embodiments, haNK® cells can also be activated by contacting an antibody and its target cells, wherein the contact results in ADCC. In some embodiments, the antibody is Rituximab (anti-CD20 antibody) and the target cells are DOHH-2 cells. In some embodiments, the antibody is Herceptin (anti-HER2 antibody) and the target cells are the SKBR3 cells. In some embodiments, the effector to target ratio is within the range from 1:1 to 1:10, e.g., 1:1, 1:2, or 1:4. In some embodiments, after the ADCC, the CD16 expression on haNK® cells, e.g., IL2 Dependent haNK® cells, decreased no more than 50%, e.g., no more than 40%, no more than 30%, no more than 25%, no more than 20%, no more than 10% as compared to the haNK003 cells before the ADCC. In some embodiments, the percentage of haNK® cells, e.g., IL2 Dependent haNK® cells, in the population that are positive for CD16 decreases no more than 20%, or no more than 10% as compared to the cells in the population before the ADCC.

Accordingly, this disclosure also provides methods of producing a population of modified NK-92® cells that are capable of maintaining expression of CD16 during activation, wherein the method comprises introducing CD16 (SEQ ID NO: 2), but not IL-2, into NK-92® cells, wherein the expression of CD16 on the activated modified NK-92® cells is no less than 50% of the CD16 expression on the modified NK-92® cells before the activation.

In some embodiments, the haNK® cells (e.g., IL-2 dependent haNK® cells) that have been activated maintain a steady state of cytotoxicity for at least 24 hours from the inititation of the activation. The cytotoxicity of the cells can be measured using methods well known in the art. In some embodiments, the cytotoxity is a direct cytotoxicity. In some embodiments, the cytotoxicity is ADCC. Maintaining a steady state of cytotoxicity during a period of time refers to that the ability of the cells to lyse target cells remain substantially the same during a reference time period. In some cases, maintaining a steady state of cytotoxicity is reflected in that the under the same assay conditions, the percentage of target cells that are lysed by the effector cells at the end of the reference time period is at least 70%, e.g., at least 75%, at least 80%, at least 85%, at least 90%, or at least 95% of the percentage of the target cells lysed by the effector cells at the beginning of the reference time period.

### Additional Transgenes

In some embodiments, the modified NK-92® cells, e.g. IL2 Dependent haNK® cells, are further engineered to express a chimeric antigen receptor (CAR) on the cell surface. Optionally, the CAR is specific for a tumor-specific antigen. Tumor-specific antigens are described, by way of non-limiting example, in US 2013/0189268; WO 1999024566 A1; U.S. Pat. No. 7,098,008; and WO 2000020460 A1, each of which is incorporated herein by reference in its entirety. Tumor-specific antigens include, without limitation, NKG2D, CS1, GD2, CD138, EpCAM, EBNA3C, GPA7, CD244, CA-125, ETA, MAGE, CAGE, BAGE, HAGE, LAGE, PAGE, NY-SEO-1, GAGE, CEA, CD52, CD30, MUC5AC, c-Met, EGFR, FAB, WT-1, PSMA, NY-ESO1, AFP, CEA, CTAG1B, CD19 and CD33. Additional non-limiting tumor-associated antigens, and the malignancies associated therewith, can be found in Table 1.

TABLE 1
Tumor-Specific Antigens and Associated Malignancies

| Target Antigen | Associated Malignancy |
|---|---|
| α-Folate Receptor | Ovarian Cancer |
| CAIX | Renal Cell Carcinoma |
| CD19 | B-cell Malignancies |
| | Chronic lymphocytic leukemia (CLL) |
| | B-cell CLL (B-CLL) |
| | Acute lymphoblastic leukemia (ALL); ALL |
| | post Hematopoietic stem cell transplantation |
| | (HSCT) |
| | Lymphoma; Refractory Follicular Lymphoma; |
| | B-cell non-Hodgkin lymphoma (B-NHL) |
| | Leukemia |
| | B-cell Malignancies post-HSCT |
| | B-lineage Lymphoid Malignancies post |
| | umbilical cord blood transplantation (UCBT) |
| CD19/CD20 | Lymphoblastic Leukemia |
| CD20 | Lymphomas |
| | B-Cell Malignancies |
| | B-cell Lymphomas |
| | Mantle Cell Lymphoma |
| | Indolent B-NHL |
| | Leukemia |
| CD22 | B-cell Malignancies |
| CD30 | Lymphomas; Hodgkin Lymphoma |
| CD33 | AML |
| CD44v7/8 | Cervical Carcinoma |
| CD138 | Multiple Myeloma |
| BCMA | lymphoma |
| Flt-3 | leukemia |
| CD123 | lymphoma, leukemia |
| CD244 | Neuroblastoma |
| CEA | Breast Cancer |
| | Colorectal Cancer |
| CS1 | Multiple Myeloma |
| EBNA3C | EBV Positive T-cells |
| EGP-2 | Multiple Malignancies |
| EGP-40 | Colorectal Cancer |
| EpCAM | Breast Carcinoma |
| ErbB2 (aka, HER2) | Colorectal Cancer |
| | Breast Cancer and Others |
| | Prostate Cancer |
| | Ovarian Cancer |
| | Tumors of Epithelial Origin |
| | Medulloblastoma |
| | Lung Malignancy |
| | Advanced Osteosarcoma |
| | Glioblastoma |
| ErbB2, 3, 4 | Breast Cancer and Others |
| FBP | Ovarian Cancer |
| Fetal Acetylcholine | Rhabdomyosarcoma |
| Receptor | |
| GD2 | Neuroblastoma |
| GD3 | Melanoma |
| GPA7 | Melanoma |
| IL-13R-a2 | Glioma |
| | Glioblastoma |
| | Medulloblastoma |
| KDR | Tumor Neovasculature |
| k-light chain | B-cell Malignancies |
| | B-NHL, CLL |
| LeY | Carcinomas |
| | Epithelial Derived Tumors |
| L1 Cell Adhesion | Neuroblastoma |
| Molecule | |
| MAGE-A1 | Melanoma |
| Mesothelin | Various Tumors |
| MUC1 | Breast Cancer; Ovarian Cancer |
| NKG2D Ligands | Various Tumors |
| Oncofetal Antigen | Various Tumors |
| (h5T4) | |
| PSCA | Prostate Carcinoma |
| PSMA | Prostate/Tumor Vasculature |
| TAA Targeted by | Various Tumors |
| mAb IgE | |
| TAG-72 | Adenocarcinomas |
| VEGF-R2 | Tumor Neovasculature |

In some embodiments, the CAR targets CD19, CD33 or PD-L1.

In examples, variant polypeptides are made using methods known in the art such as oligonucleotide-mediated (site-directed) mutagenesis, alanine scanning, and PCR mutagenesis. Site direct mutagenesis (Carter, 1986; Zoller and Smith, 1987), cassette mutagenesis, restriction selection mutagenesis (Wells et al., 1985) or other known techniques can be performed on the cloned DNA to produce CD16 variants (Ausubel, 2002; Sambrook and Russell, 2001).

Optionally, the CAR targets an antigen associated with a specific cancer type. Optionally, the cancer is selected from the group consisting of leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia), polycythemia vera, lymphomas (e.g., Hodgkin's disease and non-Hodgkin's disease), multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

In some embodiments, a polynucleotide encoding a CAR is mutated to alter the amino acid sequence encoding for CAR without altering the function of the CAR. For example, polynucleotide substitutions leading to amino acid substitutions at “non-essential” amino acid residues can be made in the CARs disclosed above. CARs can be engineered as described, for example, in Patent Publication Nos. WO 2014039523; US 20140242701; US 20140274909; US 20130280285; and WO 2014099671, each of which is incorporated herein by reference in its entirety. Optionally, the CAR is a CD19 CAR, a CD33 CAR or CSPG-4 CAR.

### Additional Modifications—Suicide Gene

In some embodiments, the modified NK-92® cells, e.g., the IL2 Dependent haNK® cells, are further engineered to incorporate a suicide gene. The term “suicide gene” is one that allows for the negative selection of the cells. A suicide gene is used as a safety system, allowing the cells expressing the gene to be killed by introduction of a selective agent. This is desirable in case the recombinant gene causes a mutation leading to uncontrolled cell growth. A number of suicide gene systems have been identified, including the herpes simplex virus thymidine kinase (TK) gene, the cytosine deaminase gene, the varicella-zoster virus thymidine kinase gene, the nitroreductase gene, the Escherichia coli gpt gene, and the E. coli Deo gene (also see, for example, Yazawa K, Fisher W E, Brunicardi F C: Current progress in suicide gene therapy for cancer. World J. Surg. 2002 July; 26(7):783-9). As used herein, the suicide gene is active in NK-92® cells. Typically, the suicide gene encodes for a protein that has no ill-effect on the cell but, in the presence of a specific compound, will kill the cell. Thus, the suicide gene is typically part of a system.

In one embodiment, the suicide gene is the thymidine kinase (TK) gene. The TK gene may be a wild-type or mutant TK gene (e.g., tk30, tk75, sr39tk). Cells expressing the TK protein can be killed using ganciclovir.

In another embodiment, the suicide gene is Cytosine deaminase which is toxic to cells in the presence of 5-fluorocytosine. Garcia-Sanchez et al. “Cytosine deaminase adenoviral vector and 5-fluorocytosine selectively reduce breast cancer cells 1 million-fold when they contaminate hematopoietic cells: a potential purging method for autologous transplantation.” Blood 1998 Jul. 15; 92(2):672-82.

In another embodiment, the suicide gene is cytochrome P450 which is toxic in the presence of ifosfamide, or cyclophosphamide. See e.g. Touati et al. “A suicide gene therapy combining the improvement of cyclophosphamide tumor cytotoxicity and the development of an anti-tumor immune response.” Curr Gene Ther. 2014; 14(3):236-46.

In another embodiment, the suicide gene is iCas9. Di Stasi, (2011) “Inducible apoptosis as a safety switch for adoptive cell therapy.” N Engl J Med 365: 1673-1683. See also Morgan, “Live and Let Die: A New Suicide Gene Therapy Moves to the Clinic” Molecular Therapy (2012); 20: 11-13. The iCas9 protein induces apoptosis in the presence of a small molecule AP1903. AP1903 is biologically inert small molecule, that has been shown in clinical studies to be well tolerated, and has been used in the context of adoptive cell therapy.

As with CD19 transgene disclosed above, these additional transgenes (e.g., CD19 CAR) can be engineered into an expression vector by any mechanism known to those of skill in the art. These additional may be engineered into the same expression vector or a different expression vector from the CD19 transgene. In preferred embodiments, the transgenes are engineered into the same vector.

### Methods of Treatment Using IL2 Dependent Hank® Cells

Also provided are methods of treating subjects with modified NK-92® cells, e.g., IL2 Dependent haNK® cells as described herein. Optionally, the subject is treated with the modified NK-92® cell and an antibody.

Modified NK-92® cells, e.g., IL2 Dependent haNK® cells, e.g., IL2 Dependent haNK® cells can be administered to a subject by absolute numbers of cells, e.g., said subject can be administered from about 1000 cells/injection to up to about 10 billion cells/injection, such as at about, at least about, or at most about, 1×10¹⁰, 1×10⁹, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) modified NK-92® cells, e.g., IL2 Dependent haNK® cells per injection, or any ranges between any two of the numbers, end points inclusive. Optionally, from 1×10⁸ to 1×10¹⁰ cells are administered to the subject. Optionally, the cells are administered one or more times weekly for one or more weeks. Optionally, the cells are administered once or twice weekly for 1, 2, 3, 4, 5, 6, 7, 8, 9, 10 or more weeks.

Optionally, subject are administered from about 1000 cells/injection/m² to up to about 10 billion cells/injection/m², such as at about, at least about, or at most about, 1×10⁸/m², 1×10⁷/m², 5×10⁷/m², 1×10⁶/m², 5×10⁶/m², 1×10⁵/m², 5×10⁵/m², 1×10⁴/m², 5×10⁴/m², 1×10³/m², 5×10³/m² (and so forth) modified NK-92® cells, e.g., IL2 Dependent haNK® cells per injection, or any ranges between any two of the numbers, end points inclusive.

Optionally, modified NK-92® cells, e.g., IL2 Dependent haNK® cells can be administered to such individual by relative numbers of cells, e.g., said individual can be administered about 1000 cells to up to about 10 billion cells per kilogram of the individual, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) modified NK-92® cells, e.g., IL2 Dependent haNK® cells per kilogram of the individual, or any ranges between any two of the numbers, end points inclusive.

Optionally, the total dose may calculated by m2 of body surface area, including about 1×10¹¹, 1×10¹⁰, 1×10⁹, 1×10⁸, 1×10⁷, per m², or any ranges between any two of the numbers, end points inclusive. Optionally, between about 1 billion and about 3 billion modified NK-92® cells, e.g., IL2 Dependent haNK® cells are administered to a patient. Optionally, the amount of modified NK-92® cells, e.g., IL2 Dependent haNK® cells, injected per dose may calculated by m2 of body surface area, including 1×10¹¹, 1×10¹⁰, 1×10⁹, 1×10⁸, 1×10⁷, per m².

The modified NK-92® cells, e.g., IL2 Dependent haNK® cells, and optionally other anticancer agents can be administered once to a patient with cancer can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive.

In one embodiment, the modified NK-92® cells, e.g., IL2 Dependent haNK® cells, are irradiated prior to administration to the patient. Irradiation of modified NK-92® cells, e.g., IL2 Dependent haNK® cells, is described, for example, in U.S. Pat. No. 8,034,332, which is incorporated herein by reference in its entirety. In one embodiment, modified NK-92® cells, e.g., IL2 Dependent haNK® cells, that have not been engineered to express a suicide gene are irradiated.

Optionally, modified NK-92® cells, e.g., IL2 Dependent haNK® cells, are administered in a composition comprising modified NK-92® cells, e.g., IL2 Dependent haNK® cells, and a medium, such as human serum or an equivalent thereof. Optionally, the medium comprises human serum albumin. Optionally, the medium comprises human plasma. Optionally, the medium comprises about 1% to about 15% human serum or human serum equivalent. Optionally, the medium comprises about 1% to about 10% human serum or human serum equivalent. Optionally, the medium comprises about 1% to about 5% human serum or human serum equivalent. Optionally, the medium comprises about 2.5% human serum or human serum equivalent. Optionally, the serum is human AB serum. Optionally, a serum substitute that is acceptable for use in human therapeutics is used instead of human serum. Such serum substitutes may be known in the art. Optionally, modified NK-92® cells, e.g., IL2 Dependent haNK® cells, are administered in a composition comprising modified NK-92® cells, e.g., IL2 Dependent haNK® cells, and an isotonic liquid solution that supports cell viability. Optionally, modified NK-92® cells, e.g., IL2 Dependent haNK® cells, are administered in a composition that has been reconstituted from a cryopreserved sample.

According to the methods provided herein, the subject is administered an effective amount of one or more of the agents provided herein. The terms effective amount and effective dosage are used interchangeably. The term effective amount is defined as any amount necessary to produce a desired physiologic response (e.g., reduction of inflammation). Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 22nd Edition, Gennaro, Editor (2012), and Pickar, Dosage Calculations (1999)).

Pharmaceutically acceptable compositions can include a variety of carriers and excipients. A variety of aqueous carriers can be used, e.g., buffered saline and the like. These solutions are sterile and generally free of undesirable matter. Suitable carriers and excipients and their formulations are described in Remington: The Science and Practice of Pharmacy, 21st Edition, David B. Troy, ed., Lippicott Williams & Wilkins (2005). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject. As used herein, the term pharmaceutically acceptable is used synonymously with physiologically acceptable and pharmacologically acceptable. A pharmaceutical composition will generally comprise agents for buffering and preservation in storage and can include buffers and carriers for appropriate delivery, depending on the route of administration.

The compositions may contain acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of cells in these formulations and/or other agents can vary and will be selected primarily based on fluid volumes, viscosities, body weight and the like in accordance with the particular mode of administration selected and the subject's needs.

Optionally, the modified NK-92® cells, e.g., IL2 Dependent haNK® cells, are administered to the subject in conjunction with one or more other treatments for the cancer being treated. Without being bound by theory, it is believed that co-treatment of a subject with modified NK-92® cells, e.g., IL2 Dependent haNK® cells, and another therapy for the cancer will allow the modified NK-92® cells, e.g., IL2 Dependent haNK® cells, and the alternative therapy to give the endogenous immune system a chance to clear the cancer that heretofore had overwhelmed such endogenous action. Optionally, two or more other treatments for the cancer being treated includes, for example, an antibody, radiation, chemotherapeutic, stem cell transplantation, or hormone therapy.

Optionally, an antibody is administered to the patient in conjunction with the modified NK-92® cells, e.g., IL2 Dependent haNK® cells. Optionally, the modified NK-92® cells, e.g., IL2 Dependent haNK® cells, and an antibody are administered to the subject together, e.g., in the same formulation; separately, e.g., in separate formulations, concurrently; or can be administered separately, e.g., on different dosing schedules or at different times of the day. When administered separately, the antibody can be administered in any suitable route, such as intravenous or oral administration.

Optionally, antibodies may be used to target cancerous cells or cells that express cancer-associated markers. A number of antibodies have been approved for the treatment of cancer, alone.

TABLE 2
Example FDA approved therapeutic monoclonal antibodies

| | Brand | | | Indication |
|---|---|---|---|---|
| Antibody | name | Company | Target | (Targeted disease) |
| Alemtuzumab | Campath ® | Genzyme | CD52 | Chronic lymphocytic |
| | | | | leukemia |
| Brentuximab | Adcetris ® | | CD30 | Anaplastic large cell |
| vedotin | | | | lymphoma (ALCL) |
| | | | | and Hodgkin lymphoma |
| Cetuximab | Erbitux ® | Bristol-Myers | epidermal growth | Colorectal cancer, Head and |
| | | Squibb/Eli | factor receptor | neck cancer |
| | | Lilly/Merck | | |
| | | KGaA | | |
| Gemtuzumab | Mylotarg ® | Wyeth | CD33 | Acute myelogenous |
| | | | | leukemia (with calicheamicin) |
| Ibritumomab | Zevalin ® | Spectrum | CD20 | Non-Hodgkin |
| tiuxetan | | Pharmaceuticals, | | lymphoma (with yttrium- |
| | | Inc. | | 90 or indium-111) |
| Ipilimumab (MD | Yervoy ® | | blocks CTLA-4 | Melanoma |
| X-101) | | | | |
| Ofatumumab | Arzerra ® | | CD20 | Chronic lymphocytic |
| | | | | leukemia |
| Palivizumab | Synagis ® | MedImmune | an epitope of the | Respiratory Syncytial Virus |
| | | | RSV F protein | |
| Panitumumab | Vectibix ® | Amgen | epidermal growth | Colorectal cancer |
| | | | factor receptor | |
| Rituximab | Rituxan ®, | Biogen | CD20 | Non-Hodgkin lymphoma |
| | Mabthera ® | Idec/Genentech | | |
| Tositumomab | Bexxar ® | GlaxoSmithKline | CD20 | Non-Hodgkin lymphoma |
| Trastuzumab | Herceptin ® | Genentech | ErbB2 | Breast cancer |
| Blinatunomab | | | bispecific CD19- | Philadelphia |
| | | | directed CD3 T-cell | chromosome-negative |
| | | | engager | relapsed or refractory B |
| | | | | cell precursor acute |
| | | | | lymphoblastic leukemia |
| | | | | (ALL) |
| Avelumamab | | | anti-PD-L1 | Non-small cell lung |
| | | | | cancer, metastatic Merkel |
| | | | | cell carcinoma; gastic |
| | | | | cancer, breast cancer, |
| | | | | ovarian cancer, bladder |
| | | | | cancer, melanoma, |
| | | | | meothelioma, including |
| | | | | metastatic or locally |
| | | | | advanced solid tumors |
| Daratumumab | | | CD38 | Multiple myeloma |
| Elotuzumab | | | a SLAMF7-directed | Multiple myeloma |
| | | | (also known as CD319) | |
| | | | immunostimulatory | |
| | | | antibody | |

Antibodies may treat cancer through a number of mechanisms. ADCC occurs when immune cells, such as NK cells, bind to antibodies that are bound to target cells through Fc receptors, such as CD16.

Accordingly, NK-92® cells that express CD16 are administered to a subject along with an effective amount of at least one monoclonal antibody directed against a specific cancer-associated protein, for example, alemtuzumab, bevacizumab, ibritumomab tiuxetan, ofatumumab, rituximab, and trastuzumab. Optionally, the monoclonal antibody is a naked monoclonal antibody, a conjugated monoclonal antibody or a bispecific monoclonal antibody. Optionally, a bispecific antibody can be used that binds the cancer cell and also binds a cell-surface protein present on the surface of NK-92® cells.

Cancer-specific antibodies bind to particular protein antigens that are expressed on the surfaces of cancer cells. NK-92® cells can be modified such that an antibody is associated with the NK-92® cell surface. Optionally, the antibody is specific for the cancer. In this way, the NK-92® cell can be specifically targeted to the cancer. Neutralizing antibodies may also be isolated. For example, a secreted glycoprotein, YKL-40, is elevated in multiple types of advanced human cancers. It is contemplated that an antibody to YKL-40 could be used to restrain tumor growth, angiogenesis and/or metastasis. See Faibish et al., (2011) Mol. Cancer Ther. 10(5):742-751.

Antibodies to cancer can be purchased from commercially available sources or can be produced by any method known in the art. For example, antibodies can be produced by obtaining B cells, bone marrow, or other samples from previously one or more patients who were infected by the cancer and recovered or were recovering when the sample was taken. Methods of identifying, screening, and growing antibodies (e.g., monoclonal antibodies) from these samples are known. For example, a phage display library can be made by isolating RNA from the sample or cells of interest, preparing cDNA from the isolated RNA, enriching the cDNA for heavy-chain and/or light-chain cDNA, and creating libraries using a phage display vector. Libraries can be prepared and screened as described, for example, in Maruyama, et al., which is incorporated herein by reference in its entirety. Antibodies can be made by recombinant methods or any other method. Isolation, screening, characterization, and production of human monoclonal antibodies are also described in Beerli, et al., PNAS (2008) 105(38):14336-14341, which is incorporated herein by reference in its entirety.

Combinations of agents or compositions can be administered either concomitantly (e.g., as a mixture), separately but simultaneously (e.g., via separate intravenous lines) or sequentially (e.g., one agent is administered first followed by administration of the second agent). Thus, the term combination is used to refer to concomitant, simultaneous, or sequential administration of two or more agents or compositions. The course of treatment is best determined on an individual basis depending on the particular characteristics of the subject and the type of treatment selected. The treatment, such as those disclosed herein, can be administered to the subject on a daily, twice daily, bi-weekly, monthly, or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

### Kits

Also disclosed are kits comprising the provided IL2 Dependent haNK® cells. Optionally, the kits further include one or more additional agents such as antibodies. The components of the kit may be contained in one or different containers such as one or more vials. The antibody may be in liquid or solid form (e.g., after lyophilization) to enhance shelf-life. If in liquid form, the components may comprise additives such as stabilizers and/or preservatives such as proline, glycine, or sucrose or other additives that enhance shelf-life.

Optionally, the kit may contain additional compounds such as therapeutically active compounds or drugs that are to be administered before, at the same time, or after administration of the IL2 Dependent haNK® cells and antibody. Examples of such compounds include vitamins, minerals, fludrocortisone, ibuprofen, lidocaine, quinidine, chemotherapeutic, and the like.

Optionally, instructions for use of the kits will include directions to use the kit components in the treatment of a cancer. The instructions may further contain information regarding how to prepare (e.g., dilute or reconstitute, in the case of freeze-dried protein) the antibody and IL2 Dependent haNK® cells (e.g., thawing and/or culturing). The instructions may further include guidance regarding the dosage and frequency of administration.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed while, specific references to each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

The examples below are intended to further illustrate certain aspects of the methods and compositions described herein, and are not intended to limit the scope of the claims.

### EXAMPLES

### Example 1. Materials

Cytofluorometric analyses of cell surface proteins as described in the Examples were performed by direct immunostaining using specific fluorophore-conjugated antibodies or corresponding isotype controls listed on the table above. Briefly, 10e5 cells were stained with the amount of antibody recommended by the manufacturer in 100 μl of flow cytometry staining buffer (PBS, 1% BSA) for 30 min, at 4° C., in the dark. Cells were washed twice with flow cytometry staining buffer, and resuspended in 200 μl of flow cytometry staining buffer. Samples were processed on a MACSQuant® 10 flow cytometer (Miltenyi Biotec) and data was analyzed using FlowJo software. Antibodies used in the Examples are shown in Table 3:

TABLE 3
Antibodies

| Antibody | Vendor | Catalog # |
|---|---|---|
| Mouse IgG1, κ Isotype Control APC-Cy7-conjugated | BD Biosciences | 555750 |
| Anti-Human CD16 clone 3G8 APC-Cy7-conjugated | BD Biosciences | 557758 |
| Anti-human CD56 PE-conjugated | BD Biosciences | 555516 |
| Anti-human CD337/NKp30 PE-conjugated | BD Biosciences | 558407 |
| Mouse IgG1, κ Isotype Control PE-conjugated | BD Biosciences | 555749 |
| Anti-Human CD3 FITC-conjugated | BD Biosciences | 555332 |
| Mouse IgG1, κ Isotype Control FITC-conjugated | BD Biosciences | 555748 |
| Anti-Human NKG2D APC-conjugated | BD Biosciences | 558071 |
| Mouse IgG1, κ Isotype Control APC-conjugated | BD Biosciences | 555751 |

### Example 2. Generation of IL2 Dependent Hank® Cells

IL2 Dependent haNK® cells were generated by genetically modifying NK-92® cells through stable transfection of aNK™ cells with a pCL20c-V176-CD16 lentivirus construct. This construct encodes for a CD16 sequence that has a valine, instead of a phenylalanine as in native CD16 polypeptide, at amino acid 176 (counting from the start codon of the full length protein), which allows for increased ADCC.

The pCL20c-V176-CD16 construct was produced based on pCL20c-Mp-CD19CAR-IRES-GFP (SEQ ID NO: 6), which is 8928 bp and comprises a CD19-CAR at position 2917-4380 bp, and an IRES at 4381-4980 bp, and a GFP at 4981-5700 bp. The plasmid was digested with KpnI, which cut at positions 2906, 4852 and 5729 to remove CD19-CAR and GFP. The restriction digest generated three fragments of sizes: 6015 (backbone), 1946 and 877 bp. The backbone fragment was purified and a double stranded oligo comprising a top strand (SEQ ID NO: 7) and a bottom strand (SEQ ID NO: 8) were ligated to the backbone produced above. The addition of this oligo introduced sites EcoRI, SphI, and NotI sites, which are non-cutters in the CD16 sequence. CD16 gene was cloned using PCR primers SEQ ID NO: 9 and SEQ ID NO: 10. The amplified CD16 polynucleotide contains a KpnI site and a NotI site at the ends, and was cloned into the engineered backbone fragment by digesting the backbone and the amplified CD16 with these two enzyme and ligation. The full nucleotide sequence of the pCL20c-V176-CD16 plasmid is shown in SEQ ID NO:11.

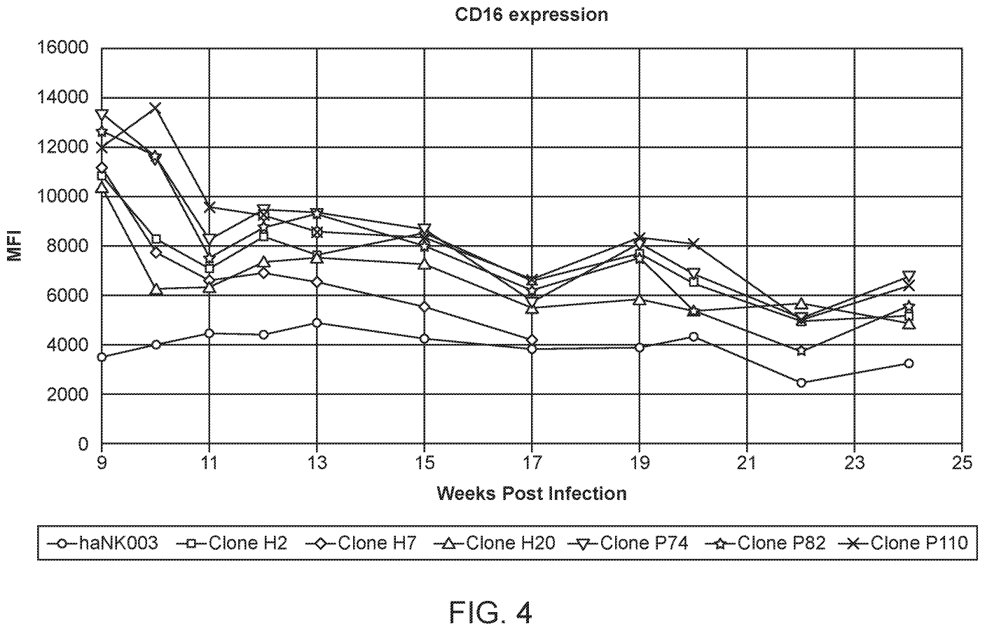

In brief, pCL20c-V176-CD16 lentivirus stocks were then produced by transfecting 7×10e6 293T cells per 10 cm petri dish with the following amount of plasmids: 7.5 μg pCL20c-V176-CD16, 5 μg pCMV-ΔR8.2, and 2.5 μg pCMV-VSV.G. The latter two plasmids are described in Naldini et al., Science April 12; 272(5259): 263-7 (1996); and Zufferey et al., Nat. Biotechnol. 1997 September; 15(9):871-5. The transfections were performed using Lipofectamine 3000 (Life Technologies, catalog #L3000-008) following manufacturer's instructions. Virus supernatants were collected 48 hour post-transfection, and concentrated 10 fold using PEG-it Virus Precipitation Solution from System Biosciences (catalog #LV810A-1). 5×10e5 aNK™ cells were infected by spinoculation (840 g for 99 min at 35° C.) with 100 μl of concentrated virus in 1 ml of final medium in a 24 well plate, in the presence of TransDux (System Biosciences, catalog #LV850A-1). V176-CD16 expressing cells were enriched using a purified anti-human CD16 Antibody (BioLegend, catalog #302002) and anti-mouse IgG MicroBeads from Miltenyi (catalog #130-048-401) following manufacturer's instructions. After enrichment, the cells were plated by limited dilution. Individual clones H2, H7, and H20 (the “H clones”), and P74, P82, and P110 (the “P clones”) were selected after grown in X-VIVO 10 medium supplemented with 5% heat-inactivated human AB serum and 500 IU/mL IL-2 for 15 days. The cells were tested for CD16 expression by flow cytometry using an antibody against CD16 conjugated to APC-Cy7 (BioLegend, catalog #302018). The CD16 expression of these individual clones during a growth period of 24 weeks post infection was monitored by flow cytometry and the results are shown in FIG. 4.

### Example 3. Generation of Hank003 Cells

haNK003 was generated by electroporating the aNK™ cells with a bicistronic plasmid-based vector containing sequences for both CD16 and IL-2. The IL-2 sequence is tagged with the endoplasmic reticulum retention signal, KDEL, to prevent IL-2 protein secretion from the endoplasmic reticulum (ER), referred to as ER IL-2, has an amino acid sequence of SEQ ID NO: 3. The polynucleotide encoding the IL-2 tagged with the endoplasmic reticulum retention signal has a nucleotide sequence of SEQ ID NO: 4.

### Transfection Plasmid

A plasmid was constructed by GeneArt AG based on provided specifications. The synthetic gene pNEUKv1_FcRIL2 (SEQ ID NO: 5) was assembled from synthetic oligonucleotides and PCR products. The fragment was cloned into the pNEUKv1_O059 vector backbone using EcoRI and NotI restriction sites. The pNEUKv1_O059 is a synthetic vector, containing an ampicillin resistance cassette. The promoter used for expression of the transgene is EF-1alpha with an SV40 polyadenylation sequence. The resulting plasmid is 5,491 base pairs (bp) in length and contains human origin sequences for CD16 and IL-2. Neither CD16 nor IL-2 have any transforming properties. The plasmid DNA was purified from transformed bacteria and its concentration was determined by UV spectroscopy. The final construct was verified by sequencing. The sequence congruence within the used restriction sites was 100%. The plasmid was made under TSE-free production conditions.

The full nucleotide sequence of the pNEUKv1_FcRIL2 plasmid is shown in SEQ ID NO:5.

To generate the haNK003 cell line, a vial of the NK-92® (aNK™) Master Cell BaNK™ (MCB) (aNK™ COA) and 250 mg of pNEUKv1_FcRIL2 plasmid were sent to EUFETS GmbH. EUFETS thawed the MCB vial and cultured the NK-92® cells to an adequate number for transfection with the plasmid. The transfected cells were grown in media with IL-2, X-VIVO 10, and 5% heat inactivated Human AB Serum for the first two days post transfection. After two days, IL-2 was no longer added to the growth media and any cells that were transfected and producing adequate amount of IL-2 continued to grow. Multiple clones were isolated by limiting dilution and preliminarily screened for phenotype and Fc Receptor expression. Six (6) clones that exhibited good viability (>70%), acceptable doubling time, expected phenotype and positive Fc Receptor expression were sent to the German Red Cross GMP Testing Laboratory (GRC) for more extensive screening and final selection of a single clone. At GRC, all clones were tested for phenotype (including Fc Receptor expression), ADCC, cytokine profile, growth characteristics, and radiation sensitivity. The selected cell line, haNK003, was used to generate the master cell bank.

Whole genome sequencing on the selected clone confirmed that the plasmid insertion site is at a single location on Chromosome 17 at position 15,654,977-15,661,403.

Multiple clones resulted from the electroporation of the aNK™ cells were selected by one round of limiting dilution. A single clone was used to establish a GMP master cell bank, haNK003.

### Example 4. Assessing CD16 Expression on IL2 Dependent Hank® Cells after PMA/Ionomycin Activation

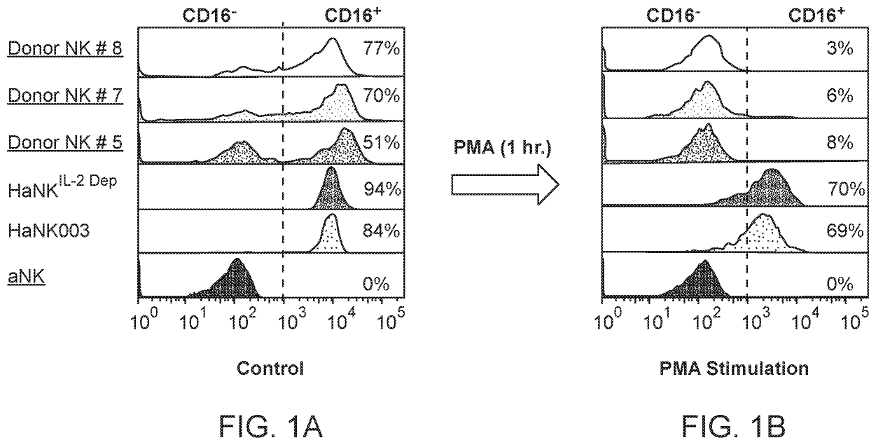

haNK003 cells and IL2 Dependent haNK® cells generated as described above, and NK cells from three donors (#5, #7, and #8) were incubated with 40 nM PMA and 669 nM ionomycin for 1 hour. CD16 expression was monitored by incubating the cells before the stimulation and cells after the stimulation with CD16-specific fluorochrome-conjugated antibodies and detecting bound antibodies by flow cytometry. The percentages of cells expressing CD16 are summarized in Table 4 and the representative, graphic illustrations are shown in FIG. 1A (before the PMA stimulation) and FIG. 1B (after the PMA stimulation).

TABLE 4
Expression of CD16 after activation by PMA/ionomycin

PMA/ionomycin activation

| | | before | after |
|---|---|---|---|
| NK cells | #8 | 77 | 3 |
| from | #7 | 70 | 6 |
| donors | #5 | 51 | 8 |

| | | |
|---|---|---|
| IL2 Dependent | 94 | 70 |
| haNK ® cells | | |
| haNK003 cells | 84 | 69 |
| aNK ™ cells | 0 | 0 |

The results show that PMA/ionomycin treatment resulted in 90%±0.06 downregulation of CD16 expression in donor NK cells, whereas the treatment resulted in only 25.5%±0.04 down-regulation in haNK003 cells and haNK-lite cells, i.e. three fold less CD16 down regulation than in donor NK cells.

### Example 5. Assessing CD16 Expression on IL2 Dependent Hank® Cells after Incubating with Target Cells

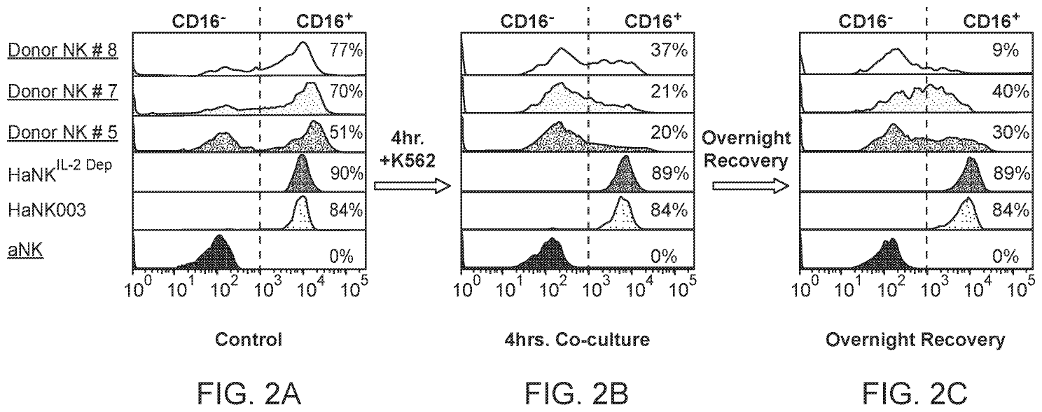

Donor NK cells from peripheral blood were obtained from Research Blood Components LLC (Boston, Mass.). MS columns (Cat. No. 130-042-201) and CD56 Microbeads, (Cat. No. 130-050-401) were obtained from Miltenyi Biotec (San Diego, Calif.). haNK003 cells, and IL2 Dependent haNK® cells were generated as described above. Donor NK cells, haNK003 cells, and IL2 Dependent haNK® cells were cultured with K562 cells (American Type Culture Collection (“ATCC”), Manassas, Va.) for 4 hours under normal co-culture condition, i.e., X-VIVO 10 culture medium supplemented with 5% human AB serum, at 37° C. for 4 h in a 5% CO2 incubator, with an effector to target ratio of 1:1 to allow complete cytotoxic killing of target cells. CD16 expression was first analyzed at the completion of the 4-hour incubation, and analyzed again after the cells were allowed to recover for additional 20 hours, i.e., the cells were analyzed at the completion of 24 hours incubation. The results are summarized in Table 5 and representative graphs shown in FIG. 2.

TABLE 5
Expression of CD16 after activation by contacting K562 cells

| | | | 4 hr | 24 hr |
|---|---|---|---|---|
| | | before | culture | culture |
| NK cells | #8 | 77 | 37 | 9 |
| from | #7 | 70 | 21 | 40 |
| donors | #5 | 51 | 20 | 30 |

| | | | |
|---|---|---|---|
| IL2 Dependent | 90 | 89 | 89 |
| haNK ® cells | | | |
| haNK003 cells | 84 | 84 | 84 |
| aNK ™ cells | 0 | 0 | 0 |

The results show that CD16 expression decreased by 61%±0.09 in donor NK cells and 4.9%±2.57 of in haNK® cells after 4 hours of co-culturing with K562. After overnight recovery (a co-culturing period of 24 hours), the downregulation of CD16 in donor NKs was about 57%, whereas the downregulation of CD16 in IL2 Dependent haNK® cells was only about 1%, i.e. close to original CD16 level (FIG. 2). This result indicates that CD16 expression in IL2 Dependent haNK® cells is stable after co-culturing with K562 cells.

### Example 6. Assessing CD16 Expression on IL2 Dependent haNK™ Cells after Antibody-Dependent Cell Mediated Cytotoxicity (ADCC)

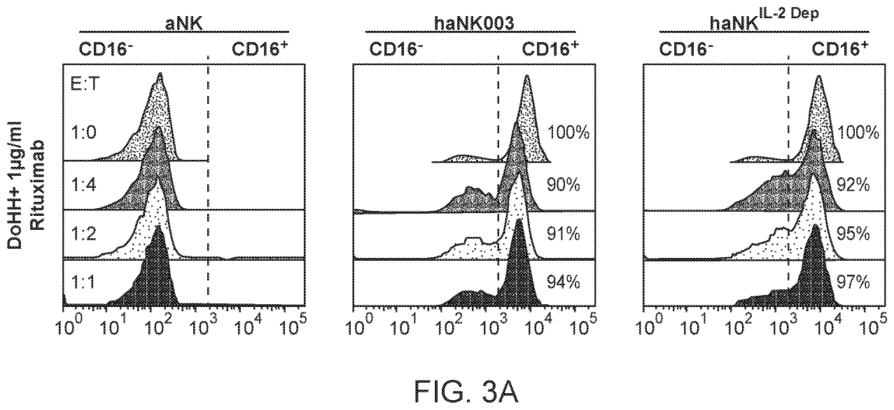

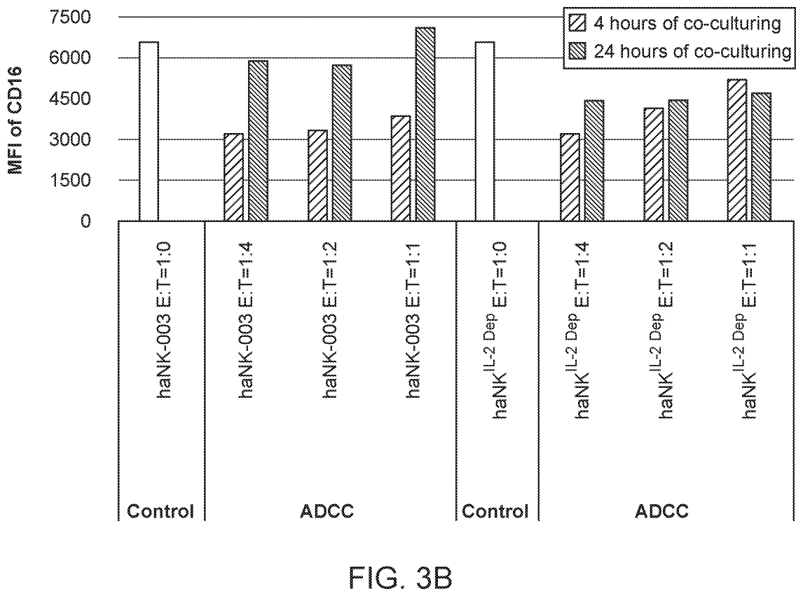

CD16 expression level was examined in haNK003 and IL2 Dependent haNK® cells after antibody-dependent cell-mediated cytotoxicity (ADCC). The ADCC was performed by incubating haNK® cells with DOHH-2 (CD20+ human lymphoma B-cell line from ATCC) in presence of 1 μg/ml Rituximab (CD20-directed cytolytic monoclonal antibody, obtained from Biogen Idec and Genentech) for 4 hours with an effector to target ratio of 1:0 (effector alone) or 1:4. CD16 expression was then measured by flow cytometry first at the end of the 4 hour incubation and then at the end of an additional 20 hour incubation. The results show that after ADCC (at the completion of 4 hour co-culturing), CD16 expression was down regulated by less than 10% in haNK® cells. See FIG. 3A, comparing the percentage of CD16+ cells to the percentage of CD16+ cells in the group where no target cells were present, i.e., E:T=1:0. FIG. 3B shows the mean fluorescence intensity (MFI) of CD16 on aNK™, haNK003, and IL2 Dependent haNK® cells after a co-culturing period of 4 hours and after a recovery period of 20 hours after the ADCC (a co-culturing period of 24 hours). The results show that CD16 was present in substantial levels in IL2 Dependent haNK® cells even after ADCC, indicating that CD16 expression in haNK® cells was highly stable in IL2 Dependent haNK® cells.

### Example 7. Phenotyping

Flow cytometry analysis were conducted to measure the surface expression of various NK cell specific markers, including CD3, CD56, CD16, CD337, CD54, and NKG2D of the IL2 Dependent haNK® clones. The results are shown in Table 6.

TABLE 6
Surface expression of NK cell specific markers

| | CD3 | CD56 | CD16 | CD337 | CD54 | NKG2D |
|---|---|---|---|---|---|---|
| aNK | −0.18% | 97.5% | 0.36% | 62.7% | 96.9% | 93.5% |
| haNK-003 | −0.01% | 96.8% | 96.8% | 70.9% | 95.0% | 88.4% |
| Clone H2 | −0.13% | 98.3% | 89.1% | 21.9% | 98.4% | 83.4% |
| Clone H7 | −0.34% | 96.9% | 86.8% | 58.9% | 96.2% | 96.2% |
| Clone H20 | −0.51% | 95.5% | 91.7% | 84.5% | 98.3% | 97.0% |
| Clone P74 | 0.19% | 90.2% | 73.6% | 70.6% | 92.2% | 87.4% |
| Clone P82 | 0.27% | 91.8% | 83.9% | 79.3% | 95.3% | 95.6% |
| Clone P110 | 2.20% | 90.0% | 74.3% | 81.4% | 84.8% | 85.6% |

The results show that, like haNK-003 cells, IL2 Dependent haNK® clones show positive expression of CD56, CD54 and NKG2D that is substantially similar to that of the aNK™ cells. All IL2 Dependent haNK® clones expressed CD16 in significant levels. All clones except clone H2 also showed significant level of CD337 expression.

### Example 8. Growth Properties

The aNK™ cells, haNK003 cells, the P and H clones of IL2 Dependent haNK® cells were grown in X-VIVO 10 medium supplemented with 5% heat-inactivated human AB serum and 500 IU/mL IL-2. The cells were seeded at 10e⁵ cells/ml and cell number was measured on day 3, 5, and 7 by trypan blue exclusion. The doubling time was determined based on the average of four experiments for each group and the results are shown in Table 7, below.

TABLE 7
Growth Properties

| | Doubling Time (hrs) |
|---|---|
| aNK | 34.8 ± 3.9 |
| haNK003 | 37.5 ± 4.6 |
| Clone H2 | 34.4 ± 3.9 |
| Clone H7 | 29.2 ± 0.6 |
| Clone H20 | 35.8 ± 1.4 |
| Clone P74 | 33.4 ± 4.6 |
| Clone P82 | 36.5 ± 3.5 |
| Clone P110 | 37.6 ± 5.2 |

The results show that the IL2 Dependent haNK® clones had substantially similar (e.g., clones H2, H20, P74, P82, and P110) or faster growth rate (e.g., clone H7) than that of the haNK-003 cells or the aNK™ cells.

### Example 9. Direct Cytoxicity

K562 cells were grown in RPMI-1640 medium (Gibco/Thermofisher) supplemented with 10% heat-inactivated FBS (Gibco/Thermofisher). K562 cells and effectors, haNK-003 cells or haNK® lite cells were combined at different effector to target ratio in a 96-well plate (Falcon B D, Franklin Lakes, N.J.), briefly centrifuged, and incubated in X-VIVO 10 culture medium supplemented with 5% human AB serum, at 37° C. for 4 h in a 5% CO₂ incubator. After incubation, cells were stained with propidium iodide (PI, Sigma-Aldrich) at 5 μg/ml in 1% BSA/PBS buffer and analyzed immediately by flow cytometry. Samples were processed on a MACSQuant® 10 flow cytometer (Miltenyi Biotec) and data was analyzed using FlowJo software.

Dead target cells, i.e., K562 cells, were identified as double positive for PKH67-GL and PI. Target cells and effector cells were also stained separately with PI to assess spontaneous cell lysis. Percentage of dead cells was determined by the percentage of PI within the PKH67⁺ target cell population. % Killing was calculated as follows=[% dead target cells in sample−% spontaneous dead target cells]/[100−% spontaneous dead target cells].

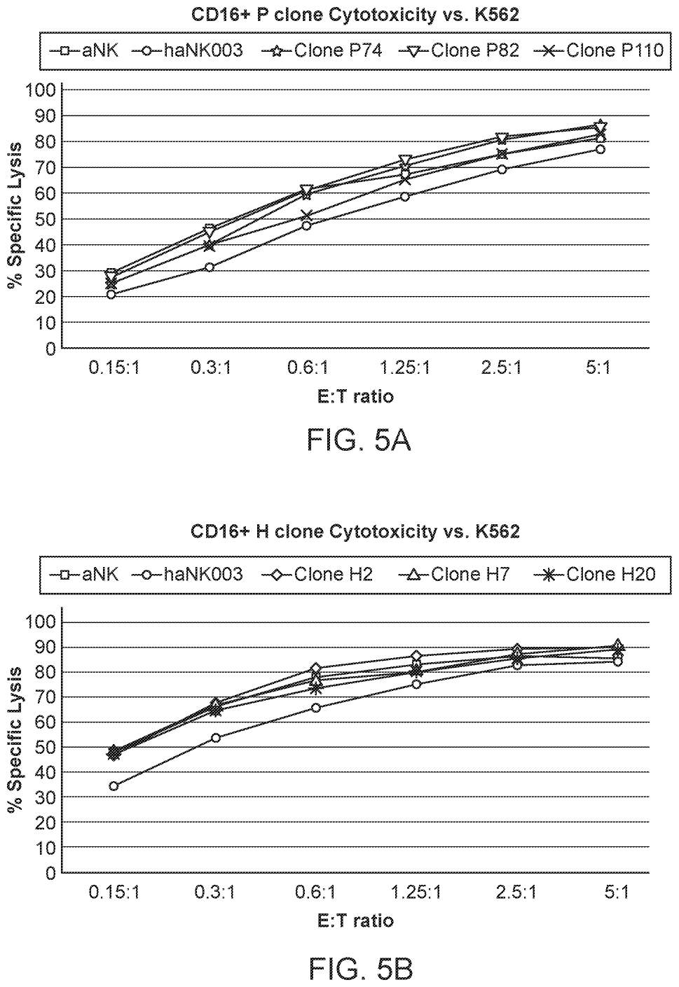

The percentage of K562 cells that were lysed by the IL2 Dependent haNK® clones were shown in FIG. 5A (P clones) and FIG. 5B (H clones). The results show that IL2 Dependent haNK® cells demonstrated substantially similar or higher cytotoxicity to the aNK™ cells when they are incubated with target cells at the same effector to target ratio. In some cases, these IL2 Dependent haNK® clones demonstrated higher cytotoxicity than that of the IL2 Independent haNK® cells.

### Example 10. ADCC

The antibody-dependent cell-mediated cytotoxicity (ADCC) of the IL2 Dependent haNK® cells on SKBR-3 cells (ATCC, Manassas, Va.) were assayed according to the methods as described in Example 9, except for an additional step of pre-incubating stained target cells with monoclonal antibodies, Herceptin or isotype control antibody (“IgG”) at different concentrations (0.001 to 1 ug/ml) prior to co-incubation with effectors. ADCC was calculated as follows=[% Killing in a reaction of E+T in the presence of mAB−% Killing in a reaction of E+T in the absence of mAb]/[100−% Killing in a reaction of E+T in the absence of mAb], (E=effector, T=target). The SKBR-3 cells were grown in RPMI-1640 medium (Gibco/Thermofisher) supplemented with 10% heat-inactivated FBS (Gibco/Thermofisher) before mixed with the effector cells.

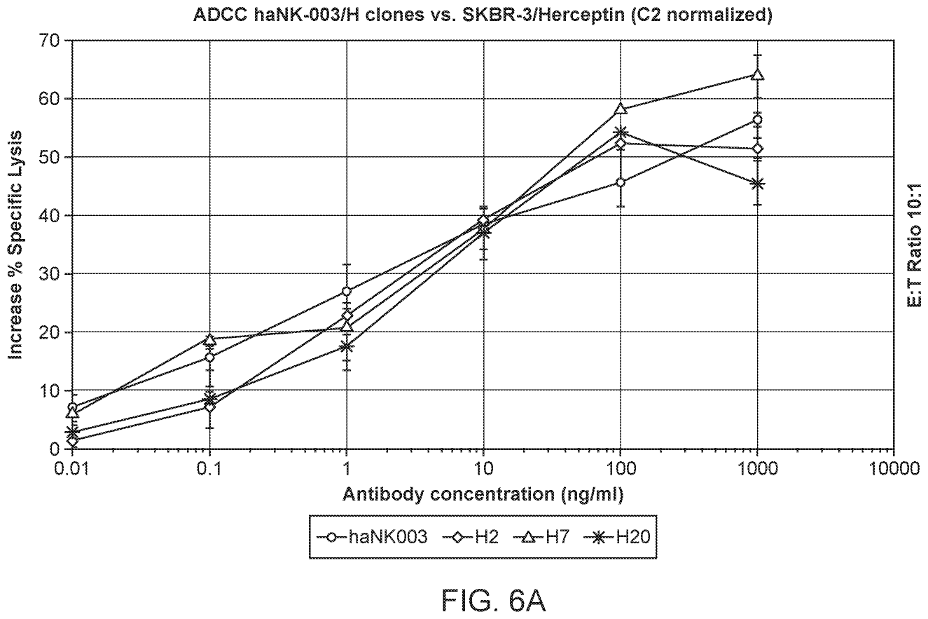

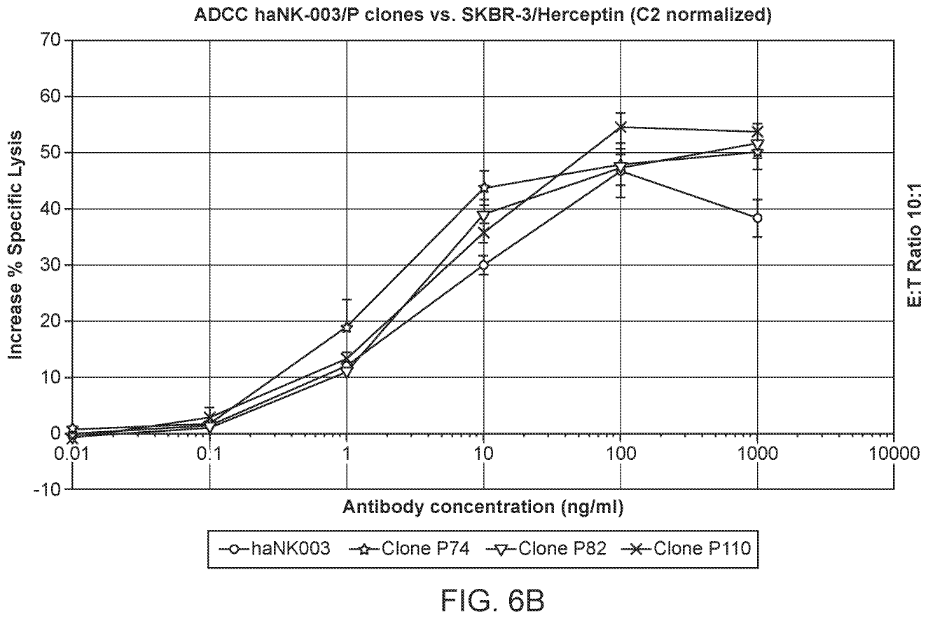

As shown in FIG. 6A and FIG. 6B, the IL2 Dependent haNK® clones demonstrated high ADCC activity and in some cases higher than that of the haNK-003 cells. For instance, the ADCC activity of the H7 clone was 40% higher than that of the haNK-003 cells, when the Herceptin were present at 100 ng/ml.

Illustrative Sequences

SEQ ID NO: 1 High Affinity Variant Immunoglobulin Gamma Fc Region Receptor
III-A amino acid sequence (full length form). The Val at position 176 is
underlined. The underlined portion in the beginning of the sequence represents
the signal peptide.
Met Trp Gln Leu Leu Leu Pro Thr Ala Leu Leu Leu Leu Val Ser Ala
Gly Met Arg Thr Glu Asp Leu Pro Lys Ala Val Val Phe Leu Glu Pro
Gln Trp Tyr Arg Val Leu Glu Lys Asp Ser Val Thr Leu Lys Cys Gln
Gly Ala Tyr Ser Pro Glu Asp Asn Ser Thr Gln Trp Phe His Asn Glu
Ser Leu Ile Ser Ser Gln Ala Ser Ser Tyr Phe Ile Asp Ala Ala Thr
Val Asp Asp Ser Gly Glu Tyr Arg Cys Gln Thr Asn Leu Ser Thr Leu
Ser Asp Pro Val Gln Leu Glu Val His Ile Gly Trp Leu Leu Leu Gln
Ala Pro Arg Trp Val Phe Lys Glu Glu Asp Pro Ile His Leu Arg Cys
His Ser Trp Lys Asn Thr Ala Leu His Lys Val Thr Tyr Leu Gln Asn
Gly Lys Gly Arg Lys Tyr Phe His His Asn Ser Asp Phe Tyr Ile Pro
Lys Ala Thr Leu Lys Asp Ser Gly Ser Tyr Phe Cys Arg Gly Leu Val
Gly Ser Lys Asn Val Ser Ser Glu Thr Val Asn Ile Thr Ile Thr Gln
Gly Leu Ala Val Ser Thr Ile Ser Ser Phe Phe Pro Pro Gly Tyr Gln
Val Ser Phe Cys Leu Val Met Val Leu Leu Phe Ala Val Asp Thr Gly
Leu Tyr Phe Ser Val Lys Thr Asn Ile Arg Ser Ser Thr Arg Asp Trp
Lys Asp His Lys Phe Lys Trp Arg Lys Asp Pro Gln Asp Lys
SEQ ID NO: 2 High Affinity Variant Immunoglobulin Gamma Fc Region Receptor
III-A nucleic acid sequence (full length form).
ATGTGGCA GCTGCTGCTG CCTACAGCTC TCCTGCTGCT GGTGTCCGCC GGCATGAGAA
CCGAGGATCT GCCTAAGGCC GTGGTGTTCC TGGAACCCCA GTGGTACAGA
GTGCTGGAAA AGGACAGCGT GACCCTGAAG TGCCAGGGCG CCTACAGCCC
CGAGGACAAT AGCACCCAGT GGTTCCACAA CGAGAGCCTG ATCAGCAGCC
AGGCCAGCAG CTACTTCATCGACGCCGCCA CCGTGGACGA CAGCGGCGAG TATAGATGCC
AGACCAACCT GAGCACCCTGAGCGACCCCG TGCAGCTGGA AGTGCACATC GGATGGCTGC
TGCTGCAGGC CCCCAGATGGGTGTTCAAAG AAGAGGACCC CATCCACCTG AGATGCCACT
CTTGGAAGAA CACCGCCCTGCACAAAGTGA CCTACCTGCA GAACGGCAAG GGCAGAAAGT
ACTTCCACCA CAACAGCGAC TTCTACATCC CCAAGGCCAC CCTGAAGGAC
TCCGGCTCCT ACTTCTGCAG AGGCCTCGTGGGCAGCAAGA ACGTGTCCAG CGAGACAGTG
AACATCACCA TCACCCAGGG CCTGGCCGTGTCTACCATCA GCAGCTTTTT CCCACCCGGC
TACCAGGTGT CCTTCTGCCT CGTGATGGTGCTGCTGTTCG CCGTGGACAC CGGCCTGTAC
TTCAGCGTGA AAACAAACAT CAGAAGCAGCACCCGGGACT GGAAGGACCA CAAGTTCAAG
TGGCGGAAGG ACCCCCAGGA CAAGTGA
SEQ ID NO: 3 ER IL-2 (ER retention signal is underlined) amino acid sequence
Met Tyr Arg Met Gln Leu Leu Ser Cys Ile Ala Leu Ser Leu Ala Leu
Val Thr Asn Ser Ala Pro Thr Ser Ser Ser Thr Lys Lys Thr Gln Leu
Gln Leu Glu His Leu Leu Leu Asp Leu Gln Met Ile Leu Asn Gly Ile
Asn Asn Tyr Lys Asn Pro Lys Leu Thr Arg Met Leu Thr Phe Lys Phe
Tyr Met Pro Lys Lys Ala Thr Glu Leu Lys His Leu Gln Cys Leu Glu
Glu Glu Leu Lys Pro Leu Glu Glu Val Leu Asn Leu Ala Gln Ser Lys
Asn Phe His Leu Arg Pro Arg Asp Leu Ile Ser Asn Ile Asn Val Ile
Val Leu Glu Leu Lys Gly Ser Glu Thr Thr Phe Met Cys Glu Tyr Ala
Asp Glu Thr Ala Thr Ile Val Glu Phe Leu Asn Arg Trp Ile Thr Phe
Cys Gln Ser Ile Ile Ser Thr Leu Thr Gly Ser Glu Lys Asp Glu Leu
SEQ ID NO: 4 ER IL-2 nucleic acid sequence
ATGTACCGGATG CAGCTGCTGA GCTGTATCGC CCTGTCTCTG GCCCTCGTGA
CCAACAGCGC CCCTACCAGC AGCAGCACCA AGAAAACCCA GCTGCAGCTG
GAACATCTGC TGCTGGACCTGCAGATGATC CTGAACGGCA TCAACAACTA CAAGAACCCC
AAGCTGACCC GGATGCTGACCTTCAAGTTC TACATGCCCA AGAAGGCCAC CGAACTGAAA
CATCTGCAGT GCCTGGAAGAGGAACTGAAG CCCCTGGAAG AAGTGCTGAA CCTGGCCCAG
AGCAAGAACT TCCACCTGAGGCCCAGGGAC CTGATCAGCA ACATCAACGT GATCGTGCTG
GAACTGAAAG GCAGCGAGACAACCTTCATG TGCGAGTACG CCGACGAGAC AGCTACCATC
GTGGAATTTC TGAACCGGTGGATCACCTTC TGCCAGAGCA TCATCAGCAC CCTGACCGGC
TCCGAGAAGG ACGAGCTGTGA
SEQ ID NO: 5 pNEUKv1_FcRIL2 plasmid

| | |
|---|---|
| 1 | TGTATTTAGA AAAATAAACA AATAGGGGTT CCGCGCACAT TTCCCCGAAA AGTGCCACCT |
| 61 | GACGTCGACG GATCGGGAGA TCTCCCGATC CCCTATGGTG CACTCTCAGT ACAATCTGCT |
| 121 | CTGATGCCGC ATAGTTAAGC CAGTATCTGC TCCCTGCTTG TGTGTTGGAG GTCGCTGAGT |
| 181 | AGTGCGCGAG CAAAATTTAA GCTACAACAA GGCAAGGCTT GACCGACAAT TGCATGAAGA |
| 241 | ATCTGCTTAG GGTTAGGCGT TTTGCGCTGC TTCGGGATCC GCTGACCAAA AGAGCACCAA |
| 301 | AGGCGCCCTG ACCTTCAGCC CCTACCTGCG CTCCGGTGCC CGTCAGTGGG CAGAGCGCAC |
| 361 | ATCGCCCACA GTCCCCGAGA AGTTGGGGGG AGGGGTCGGC AATTGAACCG GTGCCTAGAG |
| 421 | AAGGTGGCGC GGGGTAAACT GGGAAAGTGA TGTCGTGTAC TGGCTCCGCC TTTTTCCCGA |
| 481 | GGGTGGGGGA GAACCGTATA TAAGTGCAGT AGTCGCCGTG AACGTTCTTT TTCGCAACGG |
| 541 | GTTTGCCGCC AGAACACAGG TAAGTGCCGT GTGTGGTTCC CGCGGGCCTG GCCTCTTTAC |
| 601 | GGGTTATGGC CCTTGCGTGC CTTGAATTAC TTCCACCTGG CTGCAGTACG TGATTCTTGA |
| 661 | TCCCGAGCTT CGGGTTGGAA GTGGGTGGGA GAGTTCGAGG CCTTGCGCTT AAGGAGCCCC |
| 721 | TTCGCCTCGT GCTTGAGTTG AGGCCTGGCC TGGGCGCTGG GGCCGCCGCG TGCGAATCTG |
| 781 | GTGGCACCTT CGCGCCTGTC TCGCTGCTTT CGATAAGTCT CTAGCCATTT AAAATTTTTG |
| 841 | ATGACCTGCT GCGACGCTTT TTTTCTGGCA AGATAGTCTT GTAAATGCGG GCCAAGATCT |
| 901 | GCACACTGGT ATTTCGGTTT TTGGGGCCGC GGGCGGCGAC GGGGCCCGTG CGTCCCAGCG |
| 961 | CACATGTTCG GCGAGGCGGG GCCTGCGAGC GCGGCCACCG AGAATCGGAC GGGGGTAGTC |
| 1021 | TCAAGCTGGC CGGCCTGCTC TGGTGCCTGG CCTCGCGCCG CCGTGTATCG CCCCGCCCTG |
| 1081 | GGCGGCAAGG CTGGCCCGGT CGGCACCAGT TGCGTGAGCG GAAAGATGGC CGCTTCCCGG |
| 1141 | CCCTGCTGCA GGGAGCTCAA AATGGAGGAC GCGGCGCTCG GGAGAGCGGG CGGGTGAGTC |
| 1201 | ACCCACACAA AGGAAAAGGG CCTTTCCGTC CTCAGCCGTC GCTTCATGTG ACTCCACGGA |
| 1261 | GTACCGGGCG CCGTCCAGGC ACCTCGATTA GTTCTCGAGC TTTTGGAGTA CGTCGTCTTT |
| 1321 | AGGTTGGGGG GAGGGGTTTT ATGCGATGGA GTTTCCCCAC ACTGAGTGGG TGGAGACTGA |
| 1381 | AGTTAGGCCA GCTTGGCACT TGATGTAATT CTCCTTGGAA TTTGCCCTTT TTGAGTTTGG |
| 1441 | ATCTTGGTTC ATTCTCAAGC CTCAGACAGT GGTTCAAAGT TTTTTTCTTC CATTTCAGGT |
| 1501 | GTCGTGATAA TACGACTCAC TATAGGGAGA CCCAAGCTGG AATTCGCCAC CATGTGGCAG |
| 1561 | CTGCTGCTGC CTACAGCTCT CCTGCTGCTG GTGTCCGCCG GCATGAGAAC CGAGGATCTG |
| 1621 | CCTAAGGCCG TGGTGTTCCT GGAACCCCAG TGGTACAGAG TGCTGGAAAA GGACAGCGTG |
| 1681 | ACCCTGAAGT GCCAGGGCGC CTACAGCCCC GAGGACAATA GCACCCAGTG GTTCCACAAC |
| 1741 | GAGAGCCTGA TCAGCAGCCA GGCCAGCAGC TACTTCATCG ACGCCGCCAC CGTGGACGAC |
| 1801 | AGCGGCGAGT ATAGATGCCA GACCAACCTG AGCACCCTGA GCGACCCCGT GCAGCTGGAA |
| 1861 | GTGCACATCG GATGGCTGCT GCTGCAGGCC CCCAGATGGG TGTTCAAAGA AGAGGACCCC |
| 1921 | ATCCACCTGA GATGCCACTC TTGGAAGAAC ACCGCCCTGC ACAAAGTGAC CTACCTGCAG |
| 1981 | AACGGCAAGG GCAGAAAGTA CTTCCACCAC AACAGCGACT TCTACATCCC CAAGGCCACC |
| 2041 | CTGAAGGACT CCGGCTCCTA CTTCTGCAGA GGCCTCGTGG GCAGCAAGAA CGTGTCCAGC |
| 2101 | GAGACAGTGA ACATCACCAT CACCCAGGGC CTGGCCGTGT CTACCATCAG CAGCTTTTTC |
| 2161 | CCACCCGGCT ACCAGGTGTC CTTCTGCCTC GTGATGGTGC TGCTGTTCGC CGTGGACACC |
| 2221 | GGCCTGTACT TCAGCGTGAA AACAAACATC AGAAGCAGCA CCCGGGACTG GAAGGACCAC |
| 2281 | AAGTTCAAGT GGCGGAAGGA CCCCCAGGAC AAGTGAAATT CCGCCCCTCT CCCCCCCCCC |
| 2341 | CCTCTCCCTC CCCCCCCCCT AACGTTACTG GCCGAAGCCG CTTGGAATAA GGCCGGTGTG |
| 2401 | CGTTTGTCTA TATGTTATTT TCCACCATAT TGCCGTCTTT TGGCAATGTG AGGGCCCGGA |
| 2461 | AACCTGGCCC TGTCTTCTTG ACGAGCATTC CTAGGGGTCT TTCCCCTCTC GCCAAAGGAA |
| 2521 | TGCAAGGTCT GTTGAATGTC GTGAAGGAAG CAGTTCCTCT GGAAGCTTCT TGAAGACAAA |
| 2581 | CAACGTCTGT AGCGACCCTT TGCAGGCAGC GGAACCCCCC ACCTGGCGAC AGGTGCCTCT |
| 2641 | GCGGCCAAAA GCCACGTGTA TAAGATACAC CTGCAAAGGC GGCACAACCC CAGTGCCACG |
| 2701 | TTGTGAGTTG GATAGTTGTG GAAAGAGTCA AATGGCTCTC CTCAAGCGTA TTCAACAAGG |
| 2761 | GGCTGAAGGA TGCCCAGAAG GTACCCCATT GTATGGGATC TGATCTGGGG CCTCGGTGCA |
| 2821 | CATGCTTTAC ATGTGTTTAG TCGAGGTTAA AAAAACGTCT AGGCCCCCCG AACCACGGGG |
| 2881 | ACGTGGTTTT CCTTTGAAAA ACACGATAAC CGCCACCATG TACCGGATGC AGCTGCTGAG |
| 2941 | CTGTATCGCC CTGTCTCTGG CCCTCGTGAC CAACAGCGCC CCTACCAGCA GCAGCACCAA |
| 3001 | GAAAACCCAG CTGCAGCTGG AACATCTGCT GCTGGACCTG CAGATGATCC TGAACGGCAT |
| 3061 | CAACAACTAC AAGAACCCCA AGCTGACCCG GATGCTGACC TTCAAGTTCT ACATGCCCAA |
| 3121 | GAAGGCCACC GAACTGAAAC ATCTGCAGTG CCTGGAAGAG GAACTGAAGC CCCTGGAAGA |
| 3181 | AGTGCTGAAC CTGGCCCAGA GCAAGAACTT CCACCTGAGG CCCAGGGACC TGATCAGCAA |
| 3241 | CATCAACGTG ATCGTGCTGG AACTGAAAGG CAGCGAGACA ACCTTCATGT GCGAGTACGC |
| 3301 | CGACGAGACA GCTACCATCG TGGAATTTCT GAACCGGTGG ATCACCTTCT GCCAGAGCAT |
| 3361 | CATCAGCACC CTGACCGGCT CCGAGAAGGA CGAGCTGTGA GCGGCCGCCC GCTGATCAGC |
| 3421 | CTCGAACGAG ATTTCGATTC CACCGCCGCC TTCTATGAAA GGTTGGGCTT CGGAATCGTT |
| 3481 | TTCCGGGACG CCGGCTGGAT GATCCTCCAG CGCGGGGATC TCATGCTGGA GTTCTTCGCC |
| 3541 | CACCCCAACT TGTTTATTGC AGCTTATAAT GGTTACAAAT AAAGCAATAG CATCACAAAT |
| 3601 | TTCACAAATA AAGCATTTTT TTCACTGCAT TCTAGTTGTG GTTTGTCCAA ACTCATCAAT |
| 3661 | GTATCTTATC ATGTCTGTGC GGTGGGCTCT ATGGCTTCTG AGGCGGAAAG AACCAGCTGG |
| 3721 | GGCTCTAGGG GGTATCCCCG GATCCTGAGC AAAAGGCCAG CAAAAGGCCA GGAACCGTAA |
| 3781 | AAAGGCCGCG TTGCTGGCGT TTTTCCATAG GCTCCGCCCC CCTGACGAGC ATCACAAAAA |
| 3841 | TCGACGCTCA AGTCAGAGGT GGCGAAACCC GACAGGACTA TAAAGATACC AGGCGTTTCC |
| 3901 | CCCTGGAAGC TCCCTCGTGC GCTCTCCTGT TCCGACCCTG CCGCTTACCG GATACCTGTC |
| 3961 | CGCCTTTCTC CCTTCGGGAA GCGTGGCGCT TTCTCATAGC TCACGCTGTA GGTATCTCAG |
| 4021 | TTCGGTGTAG GTCGTTCGCT CCAAGCTGGG CTGTGTGCAC GAACCCCCCG TTCAGCCCGA |
| 4081 | CCGCTGCGCC TTATCCGGTA ACTATCGTCT TGAGTCCAAC CCGGTAAGAC ACGACTTATC |
| 4141 | GCCACTGGCA GCAGCCACTG GTAACAGGAT TAGCAGAGCG AGGTATGTAG GCGGTGCTAC |
| 4201 | AGAGTTCTTG AAGTGGTGGC CTAACTACGG CTACACTAGA AGAACAGTAT TTGGTATCTG |
| 4261 | CGCTCTGCTG AAGCCAGTTA CCTTCGGAAA AAGAGTTGGT AGCTCTTGAT CCGGCAAACA |
| 4321 | AACCACCGCT GGTAGCGGTG GTTTTTTTGT TTGCAAGCAG CAGATTACGC GCAGAAAAAA |
| 4381 | AGGATCTCAA GAAGATCCTT TGATCTTTTC TACGGGGTCT GACGCTCAGT GGAACGAAAA |
| 4441 | CTCACGTTAA GGGATTTTGG TCATGAGATT ATCAAAAAGG ATCTTCACCT AGATCCTTTT |
| 4501 | AAATTAAAAA TGAAGTTTTA AATCAATCTA AAGTATATAT GAGTAAACTT GGTCTGACAG |
| 4561 | TTACCAATGC TTAATCAGTG AGGCACCTAT CTCAGCGATC TGTCTATTTC GTTCATCCAT |
| 4621 | AGTTGCCTGA CTCCCCGTCG TGTAGATAAC TACGATACGG GAGGGCTTAC CATCTGGCCC |
| 4681 | CAGTGCTGCA ATGATACCGC GAGAACCACG CTCACCGGCT CCAGATTTAT CAGCAATAAA |
| 4741 | CCAGCCAGCC GGAAGGGCCG AGCGCAGAAG TGGTCCTGCA ACTTTATCCG CCTCCATCCA |
| 4801 | GTCTATTAAT TGTTGCCGGG AAGCTAGAGT AAGTAGTTCG CCAGTTAATA GTTTGCGCAA |
| 4861 | CGTTGTTGCC ATTGCTACAG GCATCGTGGT GTCACGCTCG TCGTTTGGTA TGGCTTCATT |
| 4921 | CAGCTCCGGT TCCCAACGAT CAAGGCGAGT TACATGATCC CCCATGTTGT GCAAAAAAGC |
| 4981 | GGTTAGCTCC TTCGGTCCTC CGATCGTTGT CAGAAGTAAG TTGGCCGCAG TGTTATCACT |
| 5041 | CATGGTTATG GCAGCACTGC ATAATTCTCT TACTGTCATG CCATCCGTAA GATGCTTTTC |
| 5101 | TGTGACTGGT GAGTACTCAA CCAAGTCATT CTGAGAATAG TGTATGCGGC GACCGAGTTG |
| 5161 | CTCTTGCCCG GCGTCAATAC GGGATAATAC CGCGCCACAT AGCAGAACTT TAAAAGTGCT |
| 5221 | CATCATTGGA AAACGTTCTT CGGGGCGAAA ACTCTCAAGG ATCTTACCGC TGTTGAGATC |
| 5281 | CAGTTCGATG TAACCCACTC GTGCACCCAA CTGATCTTCA GCATCTTTTA CTTTCACCAG |
| 5341 | CGTTTCTGGG TGAGCAAAAA CAGGAAGGCA AAATGCCGCA AAAAAGGGAA TAAGGGCGAC |
| 5401 | ACGGAAATGT TGAATACTCA TACTCTTCCT TTTTCAATAT TATTGAAGCA TTTATCAGGG |
| 5461 | TTATTGTCTC ATGAGCGGAT ACATATTTGA A |

SEQ ID NO: 6 pCL20 c-Mp-CD19CAR-IRES-GFP plasmid

| | |
|---|---|
| 1 | GTCGACATTG ATTATTGACT AGTTATTAAT AGTAATCAAT TACGGGGTCA TTAGTTCATA |
| 61 | GCCCATATAT GGAGTTCCGC GTTACATAAC TTACGGTAAA TGGCCCGCCT GGCTGACCGC |
| 121 | CCAACGACCC CCGCCCATTG ACGTCAATAA TGACGTATGT TCCCATAGTA ACGCCAATAG |
| 181 | GGACTTTCCA TTGACGTCAA TGGGTGGACT ATTTACGGTA AACTGCCCAC TTGGCAGTAC |
| 241 | ATCAAGTGTA TCATATGCCA AGTACGCCCC CTATTGACGT CAATGACGGT AAATGGCCCG |
| 301 | CCTGGCATTA TGCCCAGTAC ATGACCTTAT GGGACTTTCC TACTTGGCAG TACATCTACG |
| 361 | TATTAGTCAT CGCTATTACC ATGGGAGGCG TGGCCTGGGC GGGACTGGGG AGTGGCGAGC |
| 421 | CCTCAGATCC TGCATATAAG CAGCTGCTTT TTGCCTGTAC TGGGTCTCTC TGGTTAGACC |
| 481 | AGATCTGAGC CTGGGAGCTC TCTGGCTAAC TAGGGAACCC ACTGCTTAAG CCTCAATAAA |
| 541 | GCTTGCCTTG AGTGCTTCAA GTAGTGTGTG CCCGTCTGTT GTGTGACTCT GGTAACTAGA |
| 601 | GATCCCTCAG ACCCTTTTAG TCAGTGTGGA AAATCTCTAG CAGTGGCGCC CGAACAGGGA |
| 661 | CTTGAAAGCG AAAGGGAAAC CAGAGGAGCT CTCTCGACGC AGGACTCGGC TTGCTGAAGC |
| 721 | GCGCACGGCA AGAGGCGAGG GGCGGCGACT GGTGAGTACG CCAAAAATTT TGACTAGCGG |
| 781 | AGGCTAGAAG GAGAGAGATG GGTGCGAGAG CGTCAGTATT AAGCGGGGGA GAATTAGATC |
| 841 | GCGATGGGAA AAAATTCGGT TAAGGCCAGG GGGAAAGAAA AAATATAAAT TAAAACATAT |
| 901 | AGTATGGGCA AGCAGGGAGC TAGAACGATT CGCAGTTAAT CCTGGCCTGT TAGAAACATC |
| 961 | AGAAGGCTGT AGACAAATAC TGGGACAGCT ACAACCATCC CTTCAGACAG GATCAGAAGA |
| 1021 | ACTTAGATCA TTATATAATA CAGTAGCAAC CCTCTATTGT GTGCATCAAA GGATAGAGAT |
| 1081 | AAAAGACACC AAGGAAGCTT TAGACAAGAT AGAGGAAGAG CAAAACAAAA GTAAGAAAAA |
| 1141 | AGCACAGCAA GCAGCAGGAT CTTCAGACCT GGAAATTCCC TACAATCCCC AAAGTCAAGG |
| 1201 | AGTAGTAGAA TCTATGAATA AAGAATTAAA GAAAATTATA GGACAGGTAA GAGATCAGGC |
| 1261 | TGAACATCTT AAGACAGCAG TACAAATGGC AGTATTCATC CACAATTTTA AAAGAAAAGG |
| 1321 | GGGGATTGGG GGGTACAGTG CAGGGGAAAG AATAGTAGAC ATAATAGCAA CAGACATACA |
| 1381 | AACTAAAGAA TTACAAAAAC AAATTACAAA AATTCAAAAT TTTCGGGTTT ATTACAGGGA |
| 1441 | CAGCAGAAAT CCACTTTGGA AAGGACCAGC AAAGCTCCTC TGGAAAGGTG AAGGGGCAGT |
| 1501 | AGTAATACAA GATAATAGTG ACATAAAAGT AGTGCCAAGA AGAAAAGCAA AGATCATTAG |
| 1561 | GGATTATGGA AAACAGATGG CAGGTGATGA TTGTGTGGCA AGTAGACAGG ATGAGGATTA |
| 1621 | GAACATGGAA AAGTTTAGTA AAACACCATA AGGAGGAGAT ATGAGGGACA ATTGGAGAAG |
| 1681 | TGAATTATAT AAATATAAAG TAGTAAAAAT TGAACCATTA GGAGTAGCAC CCACCAAGGC |
| 1741 | AAAGAGAAGA GTGGTGCAGA GAGAAAAAAG AGCAGTGGGA ATAGGAGCTT TGTTCCTTGG |
| 1801 | GTTCTTGGGA GCAGCAGGAA GCACTATGGG CGCAGCGTCA ATGACGCTGA CGGTACAGGC |
| 1861 | CAGACAATTA TTGTCTGGTA TAGTGCAGCA GCAGAACAAT TTGCTGAGGG CTATTGAGGC |
| 1921 | GCAACAGCAT CTGTTGCAAC TCACAGTCTG GGGCATCAAG CAGCTCCAGG CAAGAATCCT |
| 1981 | GGCTGTGGAA AGATACCTAA AGGATCAACA GCTCCTGGGG ATTTGGGGTT GCTCTGGAAA |
| 2041 | ACTCATTTGC ACCACTGCTG TGCCTTGGAA TGCTAGTTGG AGTAATAAAT CTCTGGAACA |
| 2101 | GATTTGGAAT CACACGACCT GGATGGAGTG GGACAGAGAA ATTAACAATT ACACAAGCTT |
| 2161 | AATACACTCC TTAATTGAAG AATCGCAAAA CCAGCAAGAA AAGAATGAAC AAGAATTATT |
| 2221 | GGAATTAGAT AAATGGGCAA GTTTGTGGAA TTGGTTTAAC ATAACAAATT GGCTGTGGTA |
| 2281 | TATAAAATTA TTCATAATGA TAGTAGGAGG CTTGGTAGGT TTAAGAATAG TTTTTGCTGT |
| 2341 | ACTTTCTATA GTGAATAGAG TTAGGCAGGG ATATTCACCA TTATCGTTTC AGACCCACCT |
| 2401 | CCCAACCCCG AGGGGACCGA GCTCAAGCTT CGAACGCGTT AACGGGCCCA GCTTCGATAA |
| 2461 | AATAAAAGAT TTTATTTAGT CTCCAGAAAA AGGGGGGAAT GAAAGACCCC ACCTGTAGGT |
| 2521 | TTGGCAAGCT AGCTTAAGTA ACGCCATTTT GCAAGGCATG GAAAATACAT AACTGAGAAT |
| 2581 | AGAGAAGTTC AGATCAAGGT TAGGAACAGA GAGACAGCAG AATATGGGCC AAACAGGATA |
| 2641 | TCTGTGGTAA GCAGTTCCTG CCCCGGCTCA GGGCCAAGAA CAGATGGTCC CCAGATGCGG |
| 2701 | TCCCGCCCTC AGCAGTTTCT AGAGAACCAT CAGATGTTTC CAGGGTGCCC CAAGGACCTG |
| 2761 | AAAATGACCC TGTGCCTTAT TTGAACTAAC CAATCAGTTC GCTTCTCGCT TCTGTTCGCG |
| 2821 | CGCTTCTGCT CCCCGAGCTC AATAAAAGAG CCCACAACCC CTCACTCGGC GCGCCAGTCC |
| 2881 | TCCGATAGAC TGCGTCGCCC GGGTACCGGT GCCACCATGG ACTGGATCTG GCGCATCCTC |
| 2941 | TTCCTCGTCG GCGCTGCTAC CGGCGCTCAT TCGGCCCAGC CGGCCGACAT CCAGATGACA |
| 3001 | CAGACTACAT CCTCCCTGTC TGCCTCTCTG GGAGACAGAG TCACCATCAG TTGCAGGGCA |
| 3061 | AGTCAGGACA TTAGTAAATA TTTAAATTGG TATCAGCAAA AACCAGATGG AACTGTTAAA |
| 3121 | CTCCTGATCT ACCATACATC AAGATTACAC TCAGGAGTCC CATCAAGGTT CAGTGGCAGT |
| 3181 | GGGTCTGGAA CAGATTATTC TCTCACCATT AGCAACCTGG AGCAAGAAGA TATTGCCACT |
| 3241 | TACTTTTGCC AACAGGGTAA TACGCTTCCG TACACGTTCG GAGGGGGGAC CAAGCTGGAG |
| 3301 | CTGAAACGTG GTGGTGGTGG TTCTGGTGGT GGTGGTTCTG GCGGCGGCGG CTCCGGTGGT |
| 3361 | GGTGGATCCG AGGTGCAGCT GCAGCAGTCT GGACCTGGCC TGGTGGCGCC CTCACAGAGC |
| 3421 | CTGTCCGTCA CATGCACTGT CTCAGGGGTC TCATTACCCG ACTATGGTGT AAGCTGGATT |
| 3481 | CGCCAGCCTC CACGAAAGGG TCTGGAGTGG CTGGGAGTAA TATGGGGTAG TGAAACCACA |
| 3541 | TACTATAATT CAGCTCTCAA ATCCAGACTG ACCATCATCA AGGACAACTC CAAGAGCCAA |
| 3601 | GTTTTCTTAA AAATGAACAG TCTGCAAACT GATGACACAG CCATTTACTA CTGTGCCAAA |
| 3661 | CATTATTACT ACGGTGGTAG CTATGCTATG GACTACTGGG GCCAAGGGAC CACGGTCACC |
| 3721 | GTCTCCTCGG CGGCCGCTCT AGAACAGAAA CTGATCTCCG AAGAAGATCT GAACCTAGAG |
| 3781 | ATCAGCAACT CGGTGATGTA CTTCAGTTCT GTCGTGCCAG TCCTTCAGAA AGTGAACTCT |
| 3841 | ACTACTACCA AGCCAGTGCT GCGAACTCCC TCACCTGTGC ACCCTACCGG GACATCTCAG |
| 3901 | CCCCAGAGAC CAGAAGATTG TCGGCCCCGT GGCTCAGTGA AGGGGACCGG ATTGGACTTG |
| 3961 | CTAGAGGATC CCAAACTCTG CTACTTGCTA GATGGAATCC TCTTCATCTA CGGAGTCATC |
| 4021 | ATCACAGCCC TGTACCTGAG AGCAAAATTC AGCAGGAGTG CAGAGACTGC TGCCAACCTG |
| 4081 | CAGGACCCCA ACCAGCTCTA CAATGAGCTC AATCTAGGGC GAAGAGAGGA ATATGACGTC |
| 4141 | TTGGAGAAGA AGCGGGCTCG GGATCCAGAG ATGGGAGGCA AACAGCAGAG GAGGAGGAAC |
| 4201 | CCCCAGGAAG GCGTATACAA TGCACTGCAG AAAGACAAGA TGGCAGAAGC CTACAGTGAG |
| 4261 | ATCGGCACAA AAGGCGAGAG GCGGAGAGGC AAGGGGCACG ATGGCCTTTA CCAGGGTCTC |
| 4321 | AGCACTGCCA CCAAGGACAC CTATGATGCC CTGCATATGC AGACCCTGGC CCCTCGCTAA |
| 4381 | CCGCGGACAT GTACAGAGCT CGAGCGGGAT CAATTCCGCC CCCCCCCTAA CGTTACTGGC |
| 4441 | CGAAGCCGCT TGGAATAAGG CCGGTGTGCG TTTGTCTATA TGTTATTTTC CACCATATTG |
| 4501 | CCGTCTTTTG GCAATGTGAG GGCCCGGAAA CCTGGCCCTG TCTTCTTGAC GAGCATTCCT |
| 4561 | AGGGGTCTTT CCCCTCTCGC CAAAGGAATG CAAGGTCTGT TGAATGTCGT GAAGGAAGCA |
| 4621 | GTTCCTCTGG AAGCTTCTTG AAGACAAACA ACGTCTGTAG CGACCCTTTG CAGGCAGCGG |
| 4681 | AACCCCCCAC CTGGCGACAG GTGCCTCTGC GGCCAAAAGC CACGTGTATA AGATACACCT |
| 4741 | GCAAAGGCGG CACAACCCCA GTGCCACGTT GTGAGTTGGA TAGTTGTGGA AAGAGTCAAA |
| 4801 | TGGCTCTCCT CAAGCGTATT CAACAAGGGG CTGAAGGATG CCCAGAAGGT ACCCCATTGT |
| 4861 | ATGGGATCTG ATCTGGGGCC TCGGTGCACA TGCTTTACAT GTGTTTAGTC GAGGTTAAAA |
| 4921 | AACGTCTAGG CCCCCCGAAC CACGGGGACG TGGTTTTCCT TTGAAAAACA CGATAATACC |
| 4981 | ATGGTGAGCA AGGGCGAGGA GCTGTTCACC GGGGTGGTGC CCATCCTGGT CGAGCTGGAC |
| 5041 | GGCGACGTAA ACGGCCACAA GTTCAGCGTG TCCGGCGAGG GCGAGGGCGA TGCCACCTAC |
| 5101 | GGCAAGCTGA CCCTGAAGTT CATCTGCACC ACCGGCAAGC TGCCCGTGCC CTGGCCCACC |
| 5161 | CTCGTGACCA CCCTGACCTA CGGCGTGCAG TGCTTCAGCC GCTACCCCGA CCACATGAAG |
| 5221 | CAGCACGACT TCTTCAAGTC CGCCATGCCC GAAGGCTACG TCCAGGAGCG CACCATCTTC |
| 5281 | TTCAAGGACG ACGGCAACTA CAAGACCCGC GCCGAGGTGA AGTTCGAGGG CGACACCCTG |
| 5341 | GTGAACCGCA TCGAGCTGAA GGGCATCGAC TTCAAGGAGG ACGGCAACAT CCTGGGGCAC |
| 5401 | AAGCTGGAGT ACAACTACAA CAGCCACAAC GTCTATATCA TGGCCGACAA GCAGAAGAAC |
| 5461 | GGCATCAAGG TGAACTTCAA GATCCGCCAC AACATCGAGG ACGGCAGCGT GCAGCTCGCC |
| 5521 | GACCACTACC AGCAGAACAC CCCCATCGGC GACGGCCCCG TGCTGCTGCC CGACAACCAC |
| 5581 | TACCTGAGCA CCCAGTCCGC CCTGAGCAAA GACCCCAACG AGAAGCGCGA TCACATGGTC |
| 5641 | CTGCTGGAGT TCGTGACCGC CGCCGGGATC ACTCTCGGCA TGGACGAGCT GTACAAGTAA |
| 5701 | AGCGGCCGCA TCGATGCCGT ATACGGTACC TTTAAGACCA ATGACTTACA AGGCAGCTGT |
| 5761 | AGATCTTAGC CACTTTTTAA AAGAAAAGGG GGGACTGGAA GGGCTAATTC ACTCCCAAAG |
| 5821 | AAGACAAGAT CTGCTTTTTG CCTGTACTGG GTCTCTCTGG TTAGACCAGA TCTGAGCCTG |
| 5881 | GGAGCTCTCT GGCTAACTAG GGAACCCACT GCTTAAGCCT CAATAAAGCT TCAGCTGCTC |
| 5941 | GAGCTAGCAG ATCTTTTTCC CTCTGCCAAA AATTATGGGG ACATCATGAA GCCCCTTGAG |
| 6001 | CATCTGACTT CTGGCTAATA AAGGAAATTT ATTTTCATTG CAATAGTGTG TTGGAATTTT |
| 6061 | TTGTGTCTCT CACTCGGAAG GACATATGGG AGGGCAAATC ATTTAAAACA TCAGAATGAG |
| 6121 | TATTTGGTTT AGAGTTTGGC AACATATGCC ATATGCTGGC TGCCATGAAC AAAGGTGGCT |
| 6181 | ATAAAGAGGT CATCAGTATA TGAAACAGCC CCCTGCTGTC CATTCCTTAT TCCATAGAAA |
| 6241 | AGCCTTGACT TGAGGTTAGA TTTTTTTTAT ATTTTGTTTT GTGTTATTTT TTTCTTTAAC |
| 6301 | ATCCCTAAAA TTTTCCTTAC ATGTTTTACT AGCCAGATTT TTCCTCCTCT CCTGACTACT |
| 6361 | CCCAGTCATA GCTGTCCCTC TTCTCTTATG AAGATCCCTC GACCTGCAGC CCAAGCTTGG |
| 6421 | CGTAATCATG GTCATAGCTG TTTCCTGTGT GAAATTGTTA TCCGCTCACA ATTCCACACA |
| 6481 | ACATACGAGC CGGAAGCATA AAGTGTAAAG CCTGGGGTGC CTAATGAGTG AGCTAACTCA |
| 6541 | CATTAATTGC GTTGCGCTCA CTGCCCGCTT TCCAGTCGGG AAACCTGTCG TGCCAGCGGA |
| 6601 | TCCGCATCTC AATTAGTCAG CAACCATAGT CCCGCCCCTA ACTCCGCCCA TCCCGCCCCT |
| 6661 | AACTCCGCCC AGTTCCGCCC ATTCTCCGCC CCATGGCTGA CTAATTTTTT TTATTTATGC |
| 6721 | AGAGGCCGAG GCCGCCTCGG CCTCTGAGCT ATTCCAGAAG TAGTGAGGAG GCTTTTTTGG |
| 6781 | AGGCCTAGGC TTTTGCAAAA AGCTAACTTG TTTATTGCAG CTTATAATGG TTACAAATAA |
| 6841 | AGCAATAGCA TCACAAATTT CACAAATAAA GCATTTTTTT CACTGCATTC TAGTTGTGGT |
| 6901 | TTGTCCAAAC TCATCAATGT ATCTTATCAT GTCTGGATCC GCTGCATTAA TGAATCGGCC |
| 6961 | AACGCGCGGG GAGAGGCGGT TTGCGTATTG GGCGCTCTTC CGCTTCCTCG CTCACTGACT |
| 7021 | CGCTGCGCTC GGTCGTTCGG CTGCGGCGAG CGGTATCAGC TCACTCAAAG GCGGTAATAC |
| 7081 | GGTTATCCAC AGAATCAGGG GATAACGCAG GAAAGAACAT GTGAGCAAAA GGCCAGCAAA |
| 7141 | AGGCCAGGAA CCGTAAAAAG GCCGCGTTGC TGGCGTTTTT CCATAGGCTC CGCCCCCCTG |
| 7201 | ACGAGCATCA CAAAAATCGA CGCTCAAGTC AGAGGTGGCG AAACCCGACA GGACTATAAA |
| 7261 | GATACCAGGC GTTTCCCCCT GGAAGCTCCC TCGTGCGCTC TCCTGTTCCG ACCCTGCCGC |
| 7321 | TTACCGGATA CCTGTCCGCC TTTCTCCCTT CGGGAAGCGT GGCGCTTTCT CAATGCTCAC |
| 7381 | GCTGTAGGTA TCTCAGTTCG GTGTAGGTCG TTCGCTCCAA GCTGGGCTGT GTGCACGAAC |
| 7441 | CCCCCGTTCA GCCCGACCGC TGCGCCTTAT CCGGTAACTA TCGTCTTGAG TCCAACCCGG |
| 7501 | TAAGACACGA CTTATCGCCA CTGGCAGCAG CCACTGGTAA CAGGATTAGC AGAGCGAGGT |
| 7561 | ATGTAGGCGG TGCTACAGAG TTCTTGAAGT GGTGGCCTAA CTACGGCTAC ACTAGAAGGA |
| 7621 | CAGTATTTGG TATCTGCGCT CTGCTGAAGC CAGTTACCTT CGGAAAAAGA GTTGGTAGCT |
| 7681 | CTTGATCCGG CAAACAAACC ACCGCTGGTA GCGGTGGTTT TTTTGTTTGC AAGCAGCAGA |
| 7741 | TTACGCGCAG AAAAAAAGGA TCTCAAGAAG ATCCTTTGAT CTTTTCTACG GGGTCTGACG |
| 7801 | CTCAGTGGAA CGAAAACTCA CGTTAAGGGA TTTTGGTCAT GAGATTATCA AAAAGGATCT |
| 7861 | TCACCTAGAT CCTTTTAAAT TAAAAATGAA GTTTTAAATC AATCTAAAGT ATATATGAGT |
| 7921 | AAACTTGGTC TGACAGTTAC CAATGCTTAA TCAGTGAGGC ACCTATCTCA GCGATCTGTC |
| 7981 | TATTTCGTTC ATCCATAGTT GCCTGACTCC CCGTCGTGTA GATAACTACG ATACGGGAGG |
| 8041 | GCTTACCATC TGGCCCCAGT GCTGCAATGA TACCGCGAGA CCCACGCTCA CCGGCTCCAG |
| 8101 | ATTTATCAGC AATAAACCAG CCAGCCGGAA GGGCCGAGCG CAGAAGTGGT CCTGCAACTT |
| 8161 | TATCCGCCTC CATCCAGTCT ATTAATTGTT GCCGGGAAGC TAGAGTAAGT AGTTCGCCAG |
| 8221 | TTAATAGTTT GCGCAACGTT GTTGCCATTG CTACAGGCAT CGTGGTGTCA CGCTCGTCGT |
| 8281 | TTGGTATGGC TTCATTCAGC TCCGGTTCCC AACGATCAAG GCGAGTTACA TGATCCCCCA |
| 8341 | TGTTGTGCAA AAAAGCGGTT AGCTCCTTCG GTCCTCCGAT CGTTGTCAGA AGTAAGTTGG |
| 8401 | CCGCAGTGTT ATCACTCATG GTTATGGCAG CACTGCATAA TTCTCTTACT GTCATGCCAT |
| 8461 | CCGTAAGATG CTTTTCTGTG ACTGGTGAGT ACTCAACCAA GTCATTCTGA GAATAGTGTA |
| 8521 | TGCGGCGACC GAGTTGCTCT TGCCCGGCGT CAATACGGGA TAATACCGCG CCACATAGCA |
| 8581 | GAACTTTAAA AGTGCTCATC ATTGGAAAAC GTTCTTCGGG GCGAAAACTC TCAAGGATCT |
| 8641 | TACCGCTGTT GAGATCCAGT TCGATGTAAC CCACTCGTGC ACCCAACTGA TCTTCAGCAT |
| 8701 | CTTTTACTTT CACCAGCGTT TCTGGGTGAG CAAAAACAGG AAGGCAAAAT GCCGCAAAAA |
| 8761 | AGGGAATAAG GGCGACACGG AAATGTTGAA TACTCATACT CTTCCTTTTT CAATATTATT |
| 8821 | GAAGCATTTA TCAGGGTTAT TGTCTCATGA GCGGATACAT ATTTGAATGT ATTTAGAAAA |
| 8881 | ATAAACAAAT AGGGGTTCCG CGCACATTTC CCCGAAAAGT GCCACCTG |

SEQ ID NO: 7 The top strand of the oligo for adding to the pCL20 MCS backbone
additional restriction sites for EcoRI, SphI and NotI.
5′ CGTAACTACCGTGAATTCATCTACAAGCATGCATTGTAGTAGCGGCCGCATCGATGCCGTAT
ACCGTAC3′
SEQ ID NO: 8 The bottom strand of the oligo for adding to the pCL20 MCS
backbone additional restriction sites for EcoRI, SphI and NotI.
5′ GGTATACGGCATCGATGCGGCCGCTACTACAATGCATGCTTGTAGATGAATTCACGGTAGTT
ACGGTAC3′
SEQ ID NO: 9 The forward primer for cloning CD16
5′ ACTTCAGGTACCGGTGCCACCATGTGGCAGCTGCTGCTG3′
SEQ ID NO: 10 The Reverse primer for cloning CD16
5′ TAGGTTGCGGCCGCTCACTTGTCCTGGGGGTCCTTC3′
SEQ ID NO: 11 The nucleotide sequence of the pCL20c-V176-CD16 plasmid

| | |
|---|---|
| 1 | GTCGACATTG ATTATTGACT AGTTATTAAT AGTAATCAAT TACGGGGTCA TTAGTTCATA |
| 61 | GCCCATATAT GGAGTTCCGC GTTACATAAC TTACGGTAAA TGGCCCGCCT GGCTGACCGC |
| 121 | CCAACGACCC CCGCCCATTG ACGTCAATAA TGACGTATGT TCCCATAGTA ACGCCAATAG |
| 181 | GGACTTTCCA TTGACGTCAA TGGGTGGACT ATTTACGGTA AACTGCCCAC TTGGCAGTAC |
| 241 | ATCAAGTGTA TCATATGCCA AGTACGCCCC CTATTGACGT CAATGACGGT AAATGGCCCG |
| 301 | CCTGGCATTA TGCCCAGTAC ATGACCTTAT GGGACTTTCC TACTTGGCAG TACATCTACG |
| 361 | TATTAGTCAT CGCTATTACC ATGGGAGGCG TGGCCTGGGC GGGACTGGGG AGTGGCGAGC |
| 421 | CCTCAGATCC TGCATATAAG CAGCTGCTTT TTGCCTGTAC TGGGTCTCTC TGGTTAGACC |
| 481 | AGATCTGAGC CTGGGAGCTC TCTGGCTAAC TAGGGAACCC ACTGCTTAAG CCTCAATAAA |
| 541 | GCTTGCCTTG AGTGCTTCAA GTAGTGTGTG CCCGTCTGTT GTGTGACTCT GGTAACTAGA |
| 601 | GATCCCTCAG ACCCTTTTAG TCAGTGTGGA AAATCTCTAG CAGTGGCGCC CGAACAGGGA |
| 661 | CTTGAAAGCG AAAGGGAAAC CAGAGGAGCT CTCTCGACGC AGGACTCGGC TTGCTGAAGC |
| 721 | GCGCACGGCA AGAGGCGAGG GGCGGCGACT GGTGAGTACG CCAAAAATTT TGACTAGCGG |
| 781 | AGGCTAGAAG GAGAGAGATG GGTGCGAGAG CGTCAGTATT AAGCGGGGGA GAATTAGATC |
| 841 | GCGATGGGAA AAAATTCGGT TAAGGCCAGG GGGAAAGAAA AAATATAAAT TAAAACATAT |
| 901 | AGTATGGGCA AGCAGGGAGC TAGAACGATT CGCAGTTAAT CCTGGCCTGT TAGAAACATC |
| 961 | AGAAGGCTGT AGACAAATAC TGGGACAGCT ACAACCATCC CTTCAGACAG GATCAGAAGA |
| 1021 | ACTTAGATCA TTATATAATA CAGTAGCAAC CCTCTATTGT GTGCATCAAA GGATAGAGAT |
| 1081 | AAAAGACACC AAGGAAGCTT TAGACAAGAT AGAGGAAGAG CAAAACAAAA GTAAGAAAAA |
| 1141 | AGCACAGCAA GCAGCAGGAT CTTCAGACCT GGAAATTCCC TACAATCCCC AAAGTCAAGG |
| 1201 | AGTAGTAGAA TCTATGAATA AAGAATTAAA GAAAATTATA GGACAGGTAA GAGATCAGGC |
| 1261 | TGAACATCTT AAGACAGCAG TACAAATGGC AGTATTCATC CACAATTTTA AAAGAAAAGG |
| 1321 | GGGGATTGGG GGGTACAGTG CAGGGGAAAG AATAGTAGAC ATAATAGCAA CAGACATACA |
| 1381 | AACTAAAGAA TTACAAAAAC AAATTACAAA AATTCAAAAT TTTCGGGTTT ATTACAGGGA |
| 1441 | CAGCAGAAAT CCACTTTGGA AAGGACCAGC AAAGCTCCTC TGGAAAGGTG AAGGGGCAGT |
| 1501 | AGTAATACAA GATAATAGTG ACATAAAAGT AGTGCCAAGA AGAAAAGCAA AGATCATTAG |
| 1561 | GGATTATGGA AAACAGATGG CAGGTGATGA TTGTGTGGCA AGTAGACAGG ATGAGGATTA |
| 1621 | GAACATGGAA AAGTTTAGTA AAACACCATA AGGAGGAGAT ATGAGGGACA ATTGGAGAAG |
| 1681 | TGAATTATAT AAATATAAAG TAGTAAAAAT TGAACCATTA GGAGTAGCAC CCACCAAGGC |
| 1741 | AAAGAGAAGA GTGGTGCAGA GAGAAAAAAG AGCAGTGGGA ATAGGAGCTT TGTTCCTTGG |
| 1801 | GTTCTTGGGA GCAGCAGGAA GCACTATGGG CGCAGCGTCA ATGACGCTGA CGGTACAGGC |
| 1861 | CAGACAATTA TTGTCTGGTA TAGTGCAGCA GCAGAACAAT TTGCTGAGGG CTATTGAGGC |
| 1921 | GCAACAGCAT CTGTTGCAAC TCACAGTCTG GGGCATCAAG CAGCTCCAGG CAAGAATCCT |
| 1981 | GGCTGTGGAA AGATACCTAA AGGATCAACA GCTCCTGGGG ATTTGGGGTT GCTCTGGAAA |
| 2041 | ACTCATTTGC ACCACTGCTG TGCCTTGGAA TGCTAGTTGG AGTAATAAAT CTCTGGAACA |
| 2101 | GATTTGGAAT CACACGACCT GGATGGAGTG GGACAGAGAA ATTAACAATT ACACAAGCTT |
| 2161 | AATACACTCC TTAATTGAAG AATCGCAAAA CCAGCAAGAA AAGAATGAAC AAGAATTATT |
| 2221 | GGAATTAGAT AAATGGGCAA GTTTGTGGAA TTGGTTTAAC ATAACAAATT GGCTGTGGTA |
| 2281 | TATAAAATTA TTCATAATGA TAGTAGGAGG CTTGGTAGGT TTAAGAATAG TTTTTGCTGT |
| 2341 | ACTTTCTATA GTGAATAGAG TTAGGCAGGG ATATTCACCA TTATCGTTTC AGACCCACCT |
| 2401 | CCCAACCCCG AGGGGACCGA GCTCAAGCTT CGAACGCGTT AACGGGCCCA GCTTCGATAA |
| 2461 | AATAAAAGAT TTTATTTAGT CTCCAGAAAA AGGGGGGAAT GAAAGACCCC ACCTGTAGGT |
| 2521 | TTGGCAAGCT AGCTTAAGTA ACGCCATTTT GCAAGGCATG GAAAATACAT AACTGAGAAT |
| 2581 | AGAGAAGTTC AGATCAAGGT TAGGAACAGA GAGACAGCAG AATATGGGCC AAACAGGATA |
| 2641 | TCTGTGGTAA GCAGTTCCTG CCCCGGCTCA GGGCCAAGAA CAGATGGTCC CCAGATGCGG |
| 2701 | TCCCGCCCTC AGCAGTTTCT AGAGAACCAT CAGATGTTTC CAGGGTGCCC CAAGGACCTG |
| 2761 | AAAATGACCC TGTGCCTTAT TTGAACTAAC CAATCAGTTC GCTTCTCGCT TCTGTTCGCG |
| 2821 | CGCTTCTGCT CCCCGAGCTC AATAAAAGAG CCCACAACCC CTCACTCGGC GCGCCAGTCC |
| 2881 | TCCGATAGAC TGCGTCGCCC GGGTACCGGT GCCACCATGT GGCAGCTGCT GCTGCCTACA |
| 2941 | GCTCTCCTGC TGCTGGTGTC CGCCGGCATG AGAACCGAGG ATCTGCCTAA GGCCGTGGTG |
| 3001 | TTCCTGGAAC CCCAGTGGTA CAGAGTGCTG GAAAAGGACA GCGTGACCCT GAAGTGCCAG |
| 3061 | GGCGCCTACA GCCCCGAGGA CAATAGCACC CAGTGGTTCC ACAACGAGAG CCTGATCAGC |
| 3121 | AGCCAGGCCA GCAGCTACTT CATCGACGCC GCCACCGTGG ACGACAGCGG CGAGTATAGA |
| 3181 | TGCCAGACCA ACCTGAGCAC CCTGAGCGAC CCCGTGCAGC TGGAAGTGCA CATCGGATGG |
| 3241 | CTGCTGCTGC AGGCCCCCAG ATGGGTGTTC AAAGAAGAGG ACCCCATCCA CCTGAGATGC |
| 3301 | CACTCTTGGA AGAACACCGC CCTGCACAAA GTGACCTACC TGCAGAACGG CAAGGGCAGA |
| 3361 | AAGTACTTCC ACCACAACAG CGACTTCTAC ATCCCCAAGG CCACCCTGAA GGACTCCGGC |
| 3421 | TCCTACTTCT GCAGAGGCCT CGTGGGCAGC AAGAACGTGT CCAGCGAGAC AGTGAACATC |
| 3481 | ACCATCACCC AGGGCCTGGC CGTGTCTACC ATCAGCAGCT TTTTCCCACC CGGCTACCAG |
| 3541 | GTGTCCTTCT GCCTCGTGAT GGTGCTGCTG TTCGCCGTGG ACACCGGCCT GTACTTCAGC |
| 3601 | GTGAAAACAA ACATCAGAAG CAGCACCCGG GACTGGAAGG ACCACAAGTT CAAGTGGCGG |
| 3661 | AAGGACCCCC AGGACAAGTG AGCGGCCGCA TCGATGCCGT ATACCGTACC TTTAAGACCA |
| 3721 | ATGACTTACA AGGCAGCTGT AGATCTTAGC CACTTTTTAA AAGAAAAGGG GGGACTGGAA |
| 3781 | GGGCTAATTC ACTCCCAAAG AAGACAAGAT CTGCTTTTTG CCTGTACTGG GTCTCTCTGG |
| 3841 | TTAGACCAGA TCTGAGCCTG GGAGCTCTCT GGCTAACTAG GGAACCCACT GCTTAAGCCT |
| 3901 | CAATAAAGCT TCAGCTGCTC GAGCTAGCAG ATCTTTTTCC CTCTGCCAAA AATTATGGGG |
| 3961 | ACATCATGAA GCCCCTTGAG CATCTGACTT CTGGCTAATA AAGGAAATTT ATTTTCATTG |
| 4021 | CAATAGTGTG TTGGAATTTT TTGTGTCTCT CACTCGGAAG GACATATGGG AGGGCAAATC |
| 4081 | ATTTAAAACA TCAGAATGAG TATTTGGTTT AGAGTTTGGC AACATATGCC ATATGCTGGC |
| 4141 | TGCCATGAAC AAAGGTGGCT ATAAAGAGGT CATCAGTATA TGAAACAGCC CCCTGCTGTC |
| 4201 | CATTCCTTAT TCCATAGAAA AGCCTTGACT TGAGGTTAGA TTTTTTTTAT ATTTTGTTTT |
| 4261 | GTGTTATTTT TTTCTTTAAC ATCCCTAAAA TTTTCCTTAC ATGTTTTACT AGCCAGATTT |
| 4321 | TTCCTCCTCT CCTGACTACT CCCAGTCATA GCTGTCCCTC TTCTCTTATG AAGATCCCTC |
| 4381 | GACCTGCAGC CCAAGCTTGG CGTAATCATG GTCATAGCTG TTTCCTGTGT GAAATTGTTA |
| 4441 | TCCGCTCACA ATTCCACACA ACATACGAGC CGGAAGCATA AAGTGTAAAG CCTGGGGTGC |
| 4501 | CTAATGAGTG AGCTAACTCA CATTAATTGC GTTGCGCTCA CTGCCCGCTT TCCAGTCGGG |
| 4561 | AAACCTGTCG TGCCAGCGGA TCCGCATCTC AATTAGTCAG CAACCATAGT CCCGCCCCTA |
| 4621 | ACTCCGCCCA TCCCGCCCCT AACTCCGCCC AGTTCCGCCC ATTCTCCGCC CCATGGCTGA |
| 4681 | CTAATTTTTT TTATTTATGC AGAGGCCGAG GCCGCCTCGG CCTCTGAGCT ATTCCAGAAG |
| 4741 | TAGTGAGGAG GCTTTTTTGG AGGCCTAGGC TTTTGCAAAA AGCTAACTTG TTTATTGCAG |
| 4801 | CTTATAATGG TTACAAATAA AGCAATAGCA TCACAAATTT CACAAATAAA GCATTTTTTT |
| 4861 | CACTGCATTC TAGTTGTGGT TTGTCCAAAC TCATCAATGT ATCTTATCAT GTCTGGATCC |
| 4921 | GCTGCATTAA TGAATCGGCC AACGCGCGGG GAGAGGCGGT TTGCGTATTG GGCGCTCTTC |
| 4981 | CGCTTCCTCG CTCACTGACT CGCTGCGCTC GGTCGTTCGG CTGCGGCGAG CGGTATCAGC |
| 5041 | TCACTCAAAG GCGGTAATAC GGTTATCCAC AGAATCAGGG GATAACGCAG GAAAGAACAT |
| 5101 | GTGAGCAAAA GGCCAGCAAA AGGCCAGGAA CCGTAAAAAG GCCGCGTTGC TGGCGTTTTT |
| 5161 | CCATAGGCTC CGCCCCCCTG ACGAGCATCA CAAAAATCGA CGCTCAAGTC AGAGGTGGCG |
| 5221 | AAACCCGACA GGACTATAAA GATACCAGGC GTTTCCCCCT GGAAGCTCCC TCGTGCGCTC |
| 5281 | TCCTGTTCCG ACCCTGCCGC TTACCGGATA CCTGTCCGCC TTTCTCCCTT CGGGAAGCGT |
| 5341 | GGCGCTTTCT CAATGCTCAC GCTGTAGGTA TCTCAGTTCG GTGTAGGTCG TTCGCTCCAA |
| 5401 | GCTGGGCTGT GTGCACGAAC CCCCCGTTCA GCCCGACCGC TGCGCCTTAT CCGGTAACTA |
| 5461 | TCGTCTTGAG TCCAACCCGG TAAGACACGA CTTATCGCCA CTGGCAGCAG CCACTGGTAA |
| 5521 | CAGGATTAGC AGAGCGAGGT ATGTAGGCGG TGCTACAGAG TTCTTGAAGT GGTGGCCTAA |
| 5581 | CTACGGCTAC ACTAGAAGGA CAGTATTTGG TATCTGCGCT CTGCTGAAGC CAGTTACCTT |
| 5641 | CGGAAAAAGA GTTGGTAGCT CTTGATCCGG CAAACAAACC ACCGCTGGTA GCGGTGGTTT |
| 5701 | TTTTGTTTGC AAGCAGCAGA TTACGCGCAG AAAAAAAGGA TCTCAAGAAG ATCCTTTGAT |
| 5761 | CTTTTCTACG GGGTCTGACG CTCAGTGGAA CGAAAACTCA CGTTAAGGGA TTTTGGTCAT |
| 5821 | GAGATTATCA AAAAGGATCT TCACCTAGAT CCTTTTAAAT TAAAAATGAA GTTTTAAATC |
| 5881 | AATCTAAAGT ATATATGAGT AAACTTGGTC TGACAGTTAC CAATGCTTAA TCAGTGAGGC |
| 5941 | ACCTATCTCA GCGATCTGTC TATTTCGTTC ATCCATAGTT GCCTGACTCC CCGTCGTGTA |
| 6001 | GATAACTACG ATACGGGAGG GCTTACCATC TGGCCCCAGT GCTGCAATGA TACCGCGAGA |
| 6061 | CCCACGCTCA CCGGCTCCAG ATTTATCAGC AATAAACCAG CCAGCCGGAA GGGCCGAGCG |
| 6121 | CAGAAGTGGT CCTGCAACTT TATCCGCCTC CATCCAGTCT ATTAATTGTT GCCGGGAAGC |
| 6181 | TAGAGTAAGT AGTTCGCCAG TTAATAGTTT GCGCAACGTT GTTGCCATTG CTACAGGCAT |
| 6241 | CGTGGTGTCA CGCTCGTCGT TTGGTATGGC TTCATTCAGC TCCGGTTCCC AACGATCAAG |
| 6301 | GCGAGTTACA TGATCCCCCA TGTTGTGCAA AAAAGCGGTT AGCTCCTTCG GTCCTCCGAT |
| 6361 | CGTTGTCAGA AGTAAGTTGG CCGCAGTGTT ATCACTCATG GTTATGGCAG CACTGCATAA |
| 6421 | TTCTCTTACT GTCATGCCAT CCGTAAGATG CTTTTCTGTG ACTGGTGAGT ACTCAACCAA |
| 6481 | GTCATTCTGA GAATAGTGTA TGCGGCGACC GAGTTGCTCT TGCCCGGCGT CAATACGGGA |
| 6541 | TAATACCGCG CCACATAGCA GAACTTTAAA AGTGCTCATC ATTGGAAAAC GTTCTTCGGG |
| 6601 | GCGAAAACTC TCAAGGATCT TACCGCTGTT GAGATCCAGT TCGATGTAAC CCACTCGTGC |
| 6661 | ACCCAACTGA TCTTCAGCAT CTTTTACTTT CACCAGCGTT TCTGGGTGAG CAAAAACAGG |
| 6721 | AAGGCAAAAT GCCGCAAAAA AGGGAATAAG GGCGACACGG AAATGTTGAA TACTCATACT |
| 6781 | CTTCCTTTTT CAATATTATT GAAGCATTTA TCAGGGTTAT TGTCTCATGA GCGGATACAT |
| 6841 | ATTTGAATGT ATTTAGAAAA ATAAACAAAT AGGGGTTCCG CGCACATTTC CCCGAAAAGT |
| 6901 | GCCACCTG |

1. A population of modified NK-92® cells expressing CD16 (SEQ ID NO:1), wherein the modified NK-92® cells do not express IL-2, and wherein the population comprises one or more of the modified NK-92® cells, wherein the expression level of CD16 does not decrease or decreases no more than 20% when the cells are activated as compared to expression level of CD16 on the cells before activation.

2. The population of modified NK-92® cells of claim 1, wherein the modified NK-92® cells comprises a nucleic acid of CD16 (SEQ ID NO:2).

3. The population of modified NK-92® cells of claim 1, wherein the modified NK-92® cells have antibody-dependent cell-mediated cytotoxicity (ADCC).

4. The population of modified NK-92® cells of claim 1, wherein the modified NK-92® cells maintain a steady state of cytotoxicity for at least 5 hours from the initiation of the activation.

5. The population of modified NK-92® cells of claim 1, wherein the cells express higher level of CD16 than NK cells from a donor.

6. The population of modified NK-92® cells of claim 1, wherein the percentage of cells that are positive for CD16 decreases no more than 20% after the cells are activated as compared to the cells before activation.

7. (canceled)

8. The population of modified NK-92® cells of claim 1,
wherein the cells are activated by a PHA stimulation, an innate pathway activation via co-incubation with K562 cells, an ADCC activation via co-incubation with Rituxan and DOHH,
wherein the cells are activated by one or more compounds selected from the group consisting of PMA, ionomycin, and LPS, or
wherein the cells are activated by contacting target tumor cells, or
wherein the cells are activated by incubating the cells with an antibody and a target cell, wherein the incubating results in ADCC.

9-10. (canceled)

11. The population of modified NK-92® cells of claim 8, wherein the target tumor cells are selected from the group consisting of K562 cells and SKBR-3 cells.

12. The population of modified NK-92® cells of claim 8, wherein the CD16 expression decreases no more than 10% as compared to the modified NK-92® cells before the activation.

13. The population of modified NK-92® cells of claim 8, wherein the CD16 expression decreases no more than 5% as compared to the modified NK-92® cells before the activation, or
wherein the percentage of cells that are positive for CD16 decreases no more than 10% after the cells are activated by contacting the target cells as compared to the cells before the activation.

14-15. (canceled)

16. The population of modified NK-92® cells of claim 8, wherein the antibody is anti-CD20 antibody and the target cell is a DOHH-2 cell, or
antibody is anti-HER2 antibody and the target cell is a SKBR3 cells.

17. (canceled)

18. The population of modified NK-92® cells of claim 8, wherein the effector to target ratio is 1:1 to 1:10.

19. The population of modified NK-92® cells of claim 1, wherein the modified NK-92® cells additionally express a chimeric antigen receptor and/or a suicide gene.

20. The population of modified NK-92® cells of claim 1, wherein the modified NK-92® cells have direct cytotoxicity of at least 60% when the effector to target ratio is 5:1.

21. The population of modified NK-92® cells of claim 1, wherein the modified NK-92® cells have ADCC activity of at least 40%.

22. A method of producing a population of modified NK-92® cells that are capable of maintaining expression of CD16 during activation, wherein the method comprises introducing CD16 (SEQ ID NO:2), but not IL-2, into NK-92® cells, wherein the expression of CD16 on the activated modified NK-92® cells is no less than 50% of the CD16 expression on the modified NK-92® cells before the activation.

23. The method of claim 22, wherein the CD16 is introduced into the NK-92® cells through lentiviral infection.

24. A kit comprising the population of modified NK-92® cells of claim 1.

25. The kit of claim 24, wherein the kit further comprises an antibody.

26. A pharmaceutical composition comprising the population of cells of claim 1 and a pharmaceutically acceptable excipient.

27. A method of treating a subject comprising administering to the subject the pharmaceutical composition of claim 26.

28. (canceled)

29. The population of modified NK-92® cells of claim 19, wherein the suicide gene is selected from the group consisting of a thymidine kinase (TK) gene, a Cytosine deaminase, cytochrome P450, and iCas9.


SEQUENCE LISTING (12 sequences)

SEQ ID NO: 1 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 2 - DNA, 765 bp, Homo sapiens
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtga

SEQ ID NO: 3 - protein, 160 aa, Unknown
  source: /note=“Description of Unknown ER IL-2 sequence”
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSEKDEL

SEQ ID NO: 4 - DNA, 483 bp, Unknown
  source: /note=“Description of Unknown ER IL-2 sequence”
atgtaccggatgcagctgctgagctgtatcgccctgtctctggccctcgtgaccaacagcgcccctaccagcagcagcaccaagaaaacccagctgcagctggaacatctgctgctggacctgcagatgatcctgaacggcatcaacaactacaagaaccccaagctgacccggatgctgaccttcaagttctacatgcccaagaaggccaccgaactgaaacatctgcagtgcctggaagaggaactgaagcccctggaagaagtgctgaacctggcccagagcaagaacttccacctgaggcccagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgaccggctccgagaaggacgagctgtga

SEQ ID NO: 5 - DNA, 5491 bp, Artificial Sequence
  source: /note=“Description of Artificial Sequence Synthetic polynucleotide”
tgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgggatccgctgaccaaaagagcaccaaaggcgccctgaccttcagcccctacctgcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgataatacgactcactatagggagacccaagctggaattcgccaccatgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtgaaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataaccgccaccatgtaccggatgcagctgctgagctgtatcgccctgtctctggccctcgtgaccaacagcgcccctaccagcagcagcaccaagaaaacccagctgcagctggaacatctgctgctggacctgcagatgatcctgaacggcatcaacaactacaagaaccccaagctgacccggatgctgaccttcaagttctacatgcccaagaaggccaccgaactgaaacatctgcagtgcctggaagaggaactgaagcccctggaagaagtgctgaacctggcccagagcaagaacttccacctgaggcccagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgaccggctccgagaaggacgagctgtgagcggccgcccgctgatcagcctcgaacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtgcggtgggctctatggcttctgaggcggaaagaaccagctggggctctagggggtatccccggatcctgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaa

SEQ ID NO: 6 - DNA, 8928 bp, Artificial Sequence
  source: /note=“Description of Artificial Sequence Synthetic polynucleotide”
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgttaacgggcccagcttcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtaccggtgccaccatggactggatctggcgcatcctcttcctcgtcggcgctgctaccggcgctcattcggcccagccggccgacatccagatgacacagactacatcctccctgtctgcctctctgggagacagagtcaccatcagttgcagggcaagtcaggacattagtaaatatttaaattggtatcagcaaaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagctgaaacgtggtggtggtggttctggtggtggtggttctggcggcggcggctccggtggtggtggatccgaggtgcagctgcagcagtctggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaagggaccacggtcaccgtctcctcggcggccgctctagaacagaaactgatctccgaagaagatctgaacctagagatcagcaactcggtgatgtacttcagttctgtcgtgccagtccttcagaaagtgaactctactactaccaagccagtgctgcgaactccctcacctgtgcaccctaccgggacatctcagccccagagaccagaagattgtcggccccgtggctcagtgaaggggaccggattggacttgctagaggatcccaaactctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaaccgcggacatgtacagagctcgagcgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcatcgatgccgtatacggtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 7 - DNA, 69 bp, Artificial Sequence
  source: /note=“Description of Artificial Sequence Synthetic oligonucleotide”
cgtaactaccgtgaattcatctacaagcatgcattgtagtagcggccgcatcgatgccgtataccgtac

SEQ ID NO: 8 - DNA, 69 bp, Artificial Sequence
  source: /note=“Description of Artificial Sequence Synthetic oligonucleotide”
ggtatacggcatcgatgcggccgctactacaatgcatgcttgtagatgaattcacggtagttacggtac

SEQ ID NO: 9 - DNA, 39 bp, Artificial Sequence
  source: /note=“Description of Artificial Sequence Synthetic primer”
acttcaggtaccggtgccaccatgtggcagctgctgctg

SEQ ID NO: 10 - DNA, 36 bp, Artificial Sequence
  source: /note=“Description of Artificial Sequence Synthetic primer”
taggttgcggccgctcacttgtcctgggggtccttc

SEQ ID NO: 11 - DNA, 6908 bp, Artificial Sequence
  source: /note=“Description of Artificial Sequence Synthetic polynucleotide”
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgttaacgggcccagcttcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtaccggtgccaccatgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtgagcggccgcatcgatgccgtataccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 12 - protein, 4 aa, Unknown
  source: /note=“Description of Unknown Endoplasmic reticulum retention signal sequence”
KDEL